SEQUENCE LISTING (35 sequences)

SEQ ID NO: 1 - protein, 15 aa, Artificial Sequence
  CDRL1 - hPD-1.09A
RASKGVSTSGYSYLH

SEQ ID NO: 2 - protein, 7 aa, Artificial Sequence
  CDRL2 - hPD-1.09A
LASYLES

SEQ ID NO: 3 - protein, 9 aa, Artificial Sequence
  CDRL3 - hPD-1.09A
QHSRDLPLT

SEQ ID NO: 4 - protein, 111 aa, Artificial Sequence [sequence omitted]

SEQ ID NO: 5 - protein, 218 aa, Artificial Sequence
  mature K09A light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  CDRH1 - hPD-1.09A
NYYMY

SEQ ID NO: 7 - protein, 17 aa, Artificial Sequence
  CDRH2 - hPD-1.09A
GINPSNGGTNFNEKFKN

SEQ ID NO: 8 - protein, 11 aa, Artificial Sequence
  CDRH3 - hPD-1.09A
RDYRFDMGFDY

SEQ ID NO: 9 - protein, 120 aa, Artificial Sequence [sequence omitted]

SEQ ID NO: 10 - protein, 447 aa, Artificial Sequence
  mature h109A heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 15 aa, Artificial Sequence
  CDRL1 - hPD-1.08A
RASKSVSTSGFSYLH

SEQ ID NO: 12 - protein, 7 aa, Artificial Sequence
  CDRL2 - hPD-1.08A
LASNLES

SEQ ID NO: 13 - protein, 9 aa, Artificial Sequence
  CDRL3 - hPD-1.08A
QHSWELPLT

SEQ ID NO: 14 - protein, 5 aa, Artificial Sequence
  CDRH1 - hPD-1.08A
SYYLY

SEQ ID NO: 15 - protein, 17 aa, Artificial Sequence
  CDRH2 - hPD-1.08A
GVNPSNGGTNFSEKFKS

SEQ ID NO: 16 - protein, 11 aa, Artificial Sequence
  CDRH3 - hPD-1.08A
RDSNYDGGFDY

SEQ ID NO: 17 - protein, 290 aa, Artificial Sequence
  mature human PD-L1 molecule
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 18 - protein, 112 aa, Artificial Sequence
  20C3 Light Chain Mature Variable Region
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCQQSYDVVTFGAGTKLELK

SEQ ID NO: 19 - protein, 122 aa, Artificial Sequence
  20C3 Heavy Chain Mature Variable Region
QVQVQQSGAELAEPGASVKMSCKASGYIFTSYWMHWLKQRPGQGLEWIGYINPSSDYNEYSEKFMDKATLTADKASTTAYMQLISLTSEDSAVYYCARSGWLVHGDYYFDYWGQGTTLTVSS

SEQ ID NO: 20 - protein, 112 aa, Artificial Sequence
  22C3 Light Chain Mature Variable Region
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 21 - protein, 122 aa, Artificial Sequence
  22C3 Heavy Chain Mature Variable Region
QVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS

SEQ ID NO: 22 - protein, 111 aa, Artificial Sequence
  mature K09A light chain variable region
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 23 - protein, 111 aa, Artificial Sequence
  mature K09A light chain variable region
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 24 - protein, 218 aa, Artificial Sequence
  mature K09A light chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 218 aa, Artificial Sequence
  mature K09A light chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 11 aa, Artificial Sequence
  Nivolumab CDRL1
RASQSVSSYLA

SEQ ID NO: 27 - protein, 7 aa, Artificial Sequence
  Nivolumab CDRL2
DASNRAT

SEQ ID NO: 28 - protein, 9 aa, Artificial Sequence
  Nivolumab CDRL3
QQSSNWPRT

SEQ ID NO: 29 - protein, 107 aa, Artificial Sequence [sequence omitted]

SEQ ID NO: 30 - protein, 214 aa, Artificial Sequence
  Nivolumab light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 5 aa, Artificial Sequence
  Nivolumab CDRH1
NSGMH

SEQ ID NO: 32 - protein, 17 aa, Artificial Sequence
  Nivolumab CDRH2
VIWYDGSKRYYADSVKG

SEQ ID NO: 33 - protein, 4 aa, Artificial Sequence
  Nivolumab CDRH3
NDDY

SEQ ID NO: 34 - protein, 113 aa, Artificial Sequence [sequence omitted]

SEQ ID NO: 35 - protein, 440 aa, Artificial Sequence
  Nivolumab heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2022/0380469 A1
(43) Pub. Date: Dec. 1, 2022

(54) METHODS FOR TREATING METASTATIC TRIPLE NEGATIVE BREAST CANCER WITH ANTI-PD-1 ANTIBODIES

(71) Applicant: Merck Sharp & Dohme Corp., Rahway, NJ (US)

(72) Inventors: Gursel Aktan, Phoenixville, PA (US); Roger Delemere Dansey, San Francisco, CA (US); Vassiliki Karantza, Bernardsville, NJ (US); Zifang Guo, Princeton, NJ (US); Jaime A. Mejia, Ambler, PA (US); Shu-Chih Su, Chalfont, PA (US); Jing Zhao, Doylestown, PA (US); Xuan Zhou, Chalfont, PA (US)

(73) Assignee: Merck Sharp & Dohme Corp., Rahway, NJ (US)

(21) Appl. No.: 17/762,215

(22) PCT Filed: Sep. 23, 2020
(86) PCT No.: PCT/US2020/052141

§ 371 Date: Mar. 21, 2022

Related U.S. Application Data
(60) Provisional application No. 62/906,906, filed on Sep. 27, 2019.
(60) Provisional application No. 63/062,125, filed on Aug. 6, 2020.

(51) Int. Cl. C07K 16/28; A61P 35/04

(52) U.S. Cl. CPC C07K 16/2818; A61P 35/04; C07K 2317/24; A61K 2039/545

(57) ABSTRACT

The present invention relates to methods for treating metastatic triple negative breast cancer in a human patient which has been identified as having a PD-L1 enriched tumor comprising administering, as monotherapy, an anti-PD-1 antibody or antigen binding fragment thereof (e.g., pembrolizumab) in specific amounts to the patient about every three or six weeks, wherein the PD-L1 enriched tumor is a tumor identified as having a CPS score of ≥10.

### FIELD OF THE INVENTION

The present invention relates to methods for treating metastatic triple negative breast cancer in a human patient which has been identified as having a PD-L1 enriched tumor comprising administering, as monotherapy, an anti-PD-1 antibody or antigen binding fragment thereof (e.g., pembrolizumab) in specific amounts to the patient about every three or six weeks, wherein the PD-L1 enriched tumor is a tumor identified as having a CPS score of≥10.

### CROSS-REFERENCE TO RELATED APPLICATIONS

This application claims priority to US provisional application U.S. 62/906,906, filed Sep. 27, 2019, and US provisional application U.S. 63/062,125, filed Aug. 6, 2020, herein incorporated by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named 24210-PCT-SEQTEXT-SEP2020, created on Aug. 17, 2020, and having a size of 33 kilobytes and is filed concurrently with the specification. The sequence listing contained in this ASCII formatted document is part of the specification and is herein incorporated by reference in its entirety.

### BACKGROUND OF THE INVENTION

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (Sharpe et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong et al., NatMed. 8(8):793-800 (2002); Yang et al. Invest Ophthalmol Vis Sci. 49: 2518-2525 (2008); Ghebeh et al. Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953 (2005); Inman et al., Cancer 109: 1499-1505 (2007); Shimauchi et al. Int. J. Cancer 121:2585-2590 (2007); Gao et al. Clin. Cancer Research 15: 971-979 (2009); Nakanishi J. Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T cells in breast cancer and melanoma (Ghebeh et al, BMC Cancer. 2008 8:5714-15 (2008); Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366: 2455-65; Garon et al. NEngl J Med 2015, 372: 2018-28; Hamid et al., NEngl J Med 2013, 369: 134-44; Robert et al., Lancet 2014, 384: 1109-17; Robert et al., NEngl J Med 2015, 372: 2521-32; Robert et al., NEngl J Med 2015, 372: 320-30; Topalian et al., NEngl J Med 2012, 366: 2443-54; Topalian et al., J Clin Oncol 2014, 32: 1020-30; Wolchok et al., NEngl J Med 2013, 369: 122-33). Immune therapies targeting the PD-1 axis include monoclonal antibodies directed to the PD-1 receptor (KEYTRUDA (pembrolizumab), Merck and Co., Inc., Kenilworth, NJ, USA and OPDIVO (nivolumab), Bristol-Myers Squibb Company, Princeton, N.J., USA) and also those that bind to the PD-L1 ligand (MPDL3280A; TECENTRIQ™ (atezolizumab), Genentech, San Francisco, Calif., USA; IMFINZI (durvalumab), AstraZeneca Pharmaceuticals LP, Wilmington, Del.; BAVENCIO (avelumab), Merck KGaA, Darmstadt, Germany). Both therapeutic approaches have demonstrated anti tumor effects in numerous cancer types.

Currently used chemotherapies for the treatment of metastatic triple-negative breast cancer (mTNBC) lead to short-term responses in a minority of patients and have considerable toxicities (Carey LA et al., J. Clin Oncol. 2012; 30: 2615-2623; Harris LN et al., Breast Cancer Research, 2006; 8:R66; and Staudacher L et al., Ann Oncol. 2011; 22: 848-856). Thus, there exists a need for additional therapies in the treatment of metastatic triple negative breast cancer.

### SUMMARY OF THE INVENTION

The present invention provides a method of treating metastatic triple negative breast cancer in a human patient which has been identified as having a PD-L1 enriched tumor, the method comprising administering to the patient, as monotherapy, an anti-PD-1 antibody, or antigen binding fragment thereof, wherein the PD-L1 enriched tumor is a tumor identified as having a CPS score of ≥10. In one aspect, the present invention provides a method of treating metastatic triple negative breast cancer (mTNBC) in a human patient identified as having a PD-L1 enriched tumor, the method comprising administering to the patient, as monotherapy, an anti-PD1 antibody, or antigen binding fragment thereof, wherein the PD-L1 enriched tumor is a tumor identified as having a CPS score of≥10 before the anti-PD1 antibody is administered.

Compositions, uses, and kits for use with the methods described herein also form part of the invention. In some embodiments of any of the methods, compositions, kits and uses described herein, the PD-L1 enriched tumor is a tumor identified as having a CPS score of ≥20. In some embodiments of any of the methods, compositions, kits and uses described herein, the anti-PD-1 antibody or antigen-binding fragment thereof comprises (a) light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 6, 7 and 8; or (b) light chain CDRs comprising a sequence of amino acids as set forth in SEQ ID NOs: 11, 12 and 13 and heavy chain CDRs comprising a sequence of amino acids as set forth in SEQ ID NOs: 14, 15 and 16. In some embodiments of any of the methods, compositions, kits and uses described herein, the anti-PD-1 antibody or antigen-binding fragment thereof comprises: (a) a heavy chain variable region comprising a sequence of amino acids as set forth in SEQ ID NO:9, or a variant of SEQ ID NO:9, and (b) a light chain variable region comprising: (i) a sequence of amino acids as set forth in SEQ ID NO:4, or a variant of SEQ ID NO:4, (ii) a sequence of amino acids as set forth in SEQ ID NO:22, or a variant of SEQ ID NO:22, or (iii) a sequence of amino acids as set forth in SEQ ID NO:23, or a variant of SEQ ID NO:23. In some embodiments of any of the methods, compositions, kits and uses described herein, the anti-PD-1 antibody or antigen-binding fragment thereof comprises a heavy chain variable region comprising a sequence of amino acids as set forth in SEQ ID NO:9 and a light chain variable region comprising a sequence of amino acids as set forth in SEQ ID NO:4. In some embodiments of any of the methods, compositions, kits and uses described herein, the anti-PD-1 antibody or antigen-binding fragment thereof is a monoclonal antibody comprising: (a) a heavy chain comprising a sequence of amino acids as set forth in SEQ ID NO:10, or a variant of SEQ ID NO:10, and (b) a light chain comprising a sequence of amino acids as set forth in SEQ ID NO:5, a variant of SEQ ID NO: 5, SEQ ID NO:24, a variant of SEQ ID NO:24, SEQ ID NO:25, or a variant of SEQ ID NO:25. In some embodiments of any of the methods, compositions, kits and uses described herein, the anti-PD-1 antibody or antigen-binding fragment thereof is a monoclonal antibody comprising a heavy chain comprising a sequence of amino acids as set forth in SEQ ID NO: 10 and a light chain comprising a sequence of amino acids as set forth in SEQ ID NO:5.

In select embodiments of the of any of the methods, compositions, kits and uses described herein, the antibody or antigen-binding fragment is pembrolizumab. In some embodiments of any of the methods and uses described herein, the method or use comprises administering (i) about 200 mg of an anti-PD-1 antibody (e.g., pembrolizumab) or antigen binding fragment thereof to the patient every approximately three weeks or (ii) about 400 mg of an anti-PD-1 antibody (e.g., pembrolizumab), or antigen binding fragment thereof, to the patient every approximately six weeks.

In some embodiments of any of the methods and uses described herein, the patient has received at least one prior systemic treatment for mTNBC before receiving anti-PD-1 monotherapy as described herein. In other embodiments of any of the methods and uses described herein, the patient has received at least two prior systemic treatments for mTNBC before receiving anti-PD-1 monotherapy as described herein. In some embodiments of any of the methods and uses described herein, the patient has disease progression following the at least one, or the at least two, prior systemic treatments. In other embodiments of any of the methods and uses described herein, the prior treatment comprises treatment with an anthracycline and/or taxane in the neoadjuvant, adjuvant, or metastatic setting. In some embodiments of any of the methods, compositions, kits and uses described herein, the anti-PD-1 antibody is an anti-PD-1 antibody, or antigen binding fragment thereof, as described herein. In some embodiments of any of the methods, compositions, kits and uses described herein, the anti-PD-1 monotherapy is pembrolizumab.

In all of the methods, compositions, kits and uses described herein, the anti-PD-1 antibody or antigen-binding fragment inhibits the binding of PD-L1 to PD-1, and preferably also inhibits the binding of PD-L2 to PD-1. In some embodiments of any of the methods, compositions, kits and uses described herein, the anti-PD-1 antibody or antigen-binding fragment is a monoclonal antibody, which specifically binds to PD-1 and blocks the binding of PD-L1 to PD-1. In particular embodiments of any of the methods and uses described herein, the anti-PD-1 antibody comprises a heavy chain and a light chain, and wherein the heavy and light chains comprise the amino acid sequences shown in FIG. 1 (SEQ ID NO:5 and SEQ ID NO: 10).

In certain embodiments of any of the methods and uses described herein, the anti-PD-1 antibody or antigen binding fragment is administered to a patient subcutaneously.

In alternative embodiments of any of the methods and uses described herein, the anti-PD-1 antibody or antigen binding fragment is administered to a patient intravenously.

### BRIEF DESCRIPTION OF THE DRAWINGS

FIG. 1 shows amino acid sequences of the light chain and heavy chain for an exemplary anti-PD-1 monoclonal antibody useful in the present invention (SEQ ID NOs:5 and 10, respectively). Light chain and heavy chain variable regions are underlined (SEQ ID NOs: 4 and 9) and CDRs are bold and boxed.

FIG. 2 shows the disposition of all randomized patients as allocated between the pembrolizumab and chemotherapy treatment groups.

FIG. 3 shows the prevalence of PD-L1 CPS Categories for the pembrolizumab and chemotherapy patient populations. 65.10% of the pembrolizumab population and 65.2% of the chemotherapy population had a PD-L1 CPS score of ≥1. 30.8% of the pembrolizumab population and 31.6% of the chemotherapy population had a PD-L1 CPS score of≥10. 18.3% of the pembrolizumab population and 16.8% of the chemotherapy population had a PD-L1 CPS score of≥20. Data cut off was Apr. 11, 2019.

FIGS. 4A-4B show the overall survival (OS) for patients having a PD-L1 CPS score of ≥1 (FIG. 4A), a PD-L1 CPS score of≥10 (FIG. 4B), a PD-L1 CPS score of≥20 (FIG. 4B), and for the intent to treat population (ITT) (FIG. 4A), for the pembrolizumab and the chemotherapy patient populations.

FIGS. 5A-5B show the progression-free survival for patients having a PD-L1 CPS score of≥1 (FIG. 5A), a PD-L1 CPS score of≥10 (FIG. 5B), a PD-L1 CPS score of≥20 (FIG. 5B), and for the intent to treat population (ITT) (FIG. 5A), for the pembrolizumab and the chemotherapy patient populations. Data cut off was Apr. 11, 2019.

FIG. 6 shows the overall response rate (ORR) (RR; RECIST v1.1, BICR) for patients having a PD-L1 CPS score of≥1, a PD-L1 CPS score of≥10, a PD-L1 CPS score of≥20, and for the intent to treat population (ITT), for the pembrolizumab monotherapy and the chemotherapy patient populations. Data cut off was Apr. 11, 2019. The ORR in the ITT population was 9.6% for pembrolizumab monotherapy and 10.6% for chemotherapy; 12.3% for pembrolizumab monotherapy and 9.4% for chemotherapy for CPS ≥1; 17.7% for pembrolizumab monotherapy and 9.2% for chemotherapy for CPS ≥10; and 26.3% for pembrolizumab monotherapy and 11.5% for chemotherapy for CPS 20.

FIGS. 7A-7B shows the duration of response for patients having PD-L1 CPS score of 1 (FIG. 7A), a PD-L1 CPS score of 10 (FIG. 7B), a PD-L1 CPS score of 20 (FIG. 7B), and for the intent to treat population (ITT) (FIG. 7A), for the pembrolizumab monotherapy and the chemotherapy patient populations.

### DETAILED DESCRIPTION OF THE INVENTION

I. Definitions and Abbreviations

As used throughout the specification and appended claims, the following abbreviations apply:
AE adverse event
BICR blinded independent central review
CDR complementarity determining region
CI confidence interval
CPS combined positive score
DOR duration of response
ECOG Eastern Cooperative Oncology Group
FFPE formalin-fixed paraffin-embedded
FR framework region
IgG immunoglobulin G
IHC immunohistochemistry or immunohistochemical
IV intravenous
LPS lymphoma proportion score
mAb monoclonal antibody
MPS modified proportion score
MRI magnetic resonance imaging
NCI CTCAE National Cancer Institute-Common Terminology Criteria for Adverse Events
ORR objective response rate
OS overall survival
PD progressive disease
PD-1 programmed death 1 (a.k.a. programmed cell death-1 and programmed death receptor 1)
PD-L1 programmed cell death 1 ligand 1
PD-L2 programmed cell death 1 ligand 2
PFS progression free survival
PK pharmacokinetic
Q2W one dose every two weeks
Q3W one dose every three weeks
Q6W one dose every six weeks
SAE serious adverse event
SC subcutaneous
VH immunoglobulin heavy chain variable region
VL immunoglobulin light chain variable region

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

Reference to “or” indicates either or both possibilities unless the context clearly dictates one of the indicated possibilities. In some cases, “and/or” was employed to highlight either or both possibilities.

As used herein, including the appended claims, the singular forms of words such as “a,” “an,” and “the,” include their corresponding plural references unless the context clearly dictates otherwise.

The term “about”, when modifying the quantity (e.g., mg) of a substance or composition, or the value of a parameter characterizing a step in a method, or the like, refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of 0.1%±0.5%±1%±2%±3%±4%±5%±6%±7%±8%±9% or ±10%.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Treat” or “treating” metastatic triple negative breast cancer, as used herein, means to administer an anti-PD-1 antibody, or antigen-binding fragment, to a subject having a mTNBC, or diagnosed with mTNBC, to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an anti-tumor immune response, decreasing the number of one or more tumor markers, halting or delaying the growth of a tumor or blood cancer or progression of disease associated with PD-1 binding to its ligands PD-L1 and/or PD-L2 (“PD-1-related disease”) such as cancer, stabilization of PD-1-related disease, inhibiting the growth or survival of tumor cells, eliminating or reducing the size of one or more cancerous lesions or tumors, decreasing the level of one or more tumor markers, ameliorating or abrogating the clinical manifestations of PD-1-related disease, reducing the severity or duration of the clinical symptoms of PD-1-related disease such as cancer, prolonging the survival of a patient relative to the expected survival in a similar untreated patient, and inducing complete or partial remission of a cancerous condition or other PD-1 related disease.

Positive therapeutic effects in cancer can be measured in a number of ways (See, W. A. Weber, J. Nucl. Med. 50:1S-IOS (2009)). For example, with respect to tumor growth inhibition, according to NCI standards, a T/C ≤42% is the minimum level of anti-tumor activity. A T/C<10% is considered a high anti-tumor activity level, with T/C (%)=Median tumor volume of the treated/Median tumor volume of the control x 100. In some embodiments, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the treatment methods, compositions and uses of the present invention may not be effective in achieving a positive therapeutic effect in every patient, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

The term “patient” (alternatively referred to as “subject” or “individual” herein) refers to a mammal (e.g., rat, mouse, dog, cat, rabbit) capable of being treated with the methods and compositions of the invention, most preferably a human. In some embodiments, the patient is an adult patient. In other embodiments, the patient is a pediatric patient.

The term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer 5 includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., ₂ₙd 15 ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences ofProteins of Immunological Interest, Kabat, et al.; National Institutes of Health, Bethesda, Md.; 5th ed.; NIH Publ. No. 91-3242 (1991); Kabat (1978) Adv.

Prot. Chem. 32:1-75; Kabat, et al., (1977) J. Biol. Chem. 252:6609-6616; Chothia, et al., (1987) JMol. Biol. 196:901-917 or Chothia, et al., (1989) Nature 342:878-883.

The term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain).

See Kabat et al. (1991) Sequences ofProteins of Immunological Interest, 5^{th} Ed. Public Health Service, National Institutes of Health, Bethesda, Md. (defining the CDR 35 regions of an antibody by sequence); see also Chothia and Lesk (1987) J Mol. Biol. 196: 901-917 (defining the CDR regions of an antibody by structure). The term “framework” or “FR” residues refers to those variable domain residues other than the hypervariable region residues defined herein as CDR residues.

Unless otherwise indicated, an “antibody fragment” or “antigen binding fragment” refers to antigen binding fragments of antibodies, i.e. antibody fragments that retain the ability to specifically bind to the antigen bound by the full-length antibody, e.g. fragments that retain one or more CDR regions. Examples of antibody binding fragments include, but are not limited to, Fab, Fab′, F(ab′)2, and Fv fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence. “Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity. “Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. “Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence.

The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The term “triple negative breast cancer” as used herein refers to a cancer that tests negative for estrogen receptors, progesterone receptors, and HER2.

The term “monotherapy” refers to the use of a single agent or one type of treatment (e.g., radiation therapy) to treat a certain disease or conditions, e.g., mTNBC. The term “anti-PD-1 monotherapy” refers to use of an anti-PD-1 antibody, or antigen binding fragment thereof, as the single agent to treat the disease or conditions, e.g., mTNBC. As a non-limiting example, the term “pembrolizumab monotherapy” means using pembrolizumab as the single agent treatment to treat the disease or condition, e.g., mTNBC. Monotherapy may follow previous lines of therapy, including, for example, one or more lines of prior treatment with an anthracycline and/or a taxane in the neoadjuvant, adjuvant, or metastatic setting, wherein the patient exhibited disease progression after such prior treatment(s). “Biotherapeutic agent” means a biological molecule, such as an antibody or fusion protein, that blocks ligand/receptor signaling in any biological pathway that supports tumor maintenance and/or growth or suppresses the anti-tumor immune response. “CDR” or “CDRs” means complementarity determining region(s) in an immunoglobulin variable region, generally defined using the Kabat numbering system. “Platinum-containing chemotherapy” (also known as platins) refers to the use of chemotherapeutic agent(s) used to treat cancer that are coordination complexes of platinum.

Platinum-containing chemotherapeutic agents are alkylating agents that crosslink DNA, resulting in ineffective DNA mismatch repair and generally leading to apoptosis. Examples of platins include cisplatin, carboplatin, and oxaliplatin.

“Chemotherapeutic agent” is a chemical compound useful in the treatment of cancer.

Classes of chemotherapeutic agents include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth.

Chemotherapeutic agents useful in the treatment methods of the present invention include cytostatic and/or cytotoxic agents.

“Chothia” means an antibody numbering system described in Al-Lazikani et al., JMB 273:927-948 (1997).

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino
Acid Substitutions

| Original | Conservative |
|---|---|
| residue | substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antigen binding fragment that consists essentially of a recited amino acid sequence may also include one or more amino acids, including substitutions of one or more amino acid residues, which do not materially affect the properties of the binding compound. “Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Diagnostic anti-PD-L monoclonal antibody” means a mAb which specifically binds to the mature form of the designated PD-L (PD-L1 or PD-L2) that is expressed on the surface of certain mammalian cells. A mature PD-L lacks the presecretory leader sequence, also referred to as leader peptide The terms “PD-L” and “mature PD-L” are used interchangeably herein, and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 17)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in WO 2014/100079. These antibodies comprise the light chain and heavy chain variable region amino acid sequences shown in Table 2 below:

TABLE 2
Monoclonal Antibodies 20C3 and 22C3

20C3 Light Chain Mature Variable Region

| | |
|---|---|
| DIVMSQSPSSLAVSAGEKVTMSCKSSQSLLNSRTRKNYLAWYQQ | SEQ ID NO: 18 |
| KPGQSPKLLIYWASTRESGVPDRFTGSGSGTDFTLTISSVQAEDLA | |
| VYYCQQSYDVVTFGAGTKLELK | |

20C3 Heavy Chain Mature Variable Region

| | |
|---|---|
| QVQVQQSGAELAEPGASVKMSCKASGYIFTSYWMHWLKQRPGQ | SEQ ID NO: 19 |
| GLEWIGYINPSSDYNEYSEKFMDKATLTADKASTTAYMQLISLTS | |
| EDSAVYYCARSGWLVHGDYYFDYWGQGTTLTVSS | |

22C3 Light Chain Mature Variable Region

| | |
|---|---|
| DIVMSQSPSSLAVSAGEKVTMTCKSSQSLLHTSTRKNYLAWYQQ | SEQ ID NO: 20 |
| KPGQSPKLLIYWASTRESGVPDRFTGSGSGTDFTLTISSVQAEDLA | |
| VYYCKQSYDWTFGAGTKLELK | |

22C3 Heavy Chain Mature Variable Region

| | |
|---|---|
| QVHLQQSGAELAKPGASVKMSCKASGYTFTSYWIHWIKQRPGQG | SEQ ID NO: 21 |
| LEWIGYINPSSGYHEYNQKFIDKATLTADRSSSTAYMHLTSLTSED | |
| SAVYYCARSGWLIHGDYYFDFWGQGTTLTVSS | |

Another anti-human PD-L1 mAb that has been reported to be useful for IHC detection of PD-L1 expression in FFPE tissue sections (Chen, B. J. et al., Clin Cancer Res 19:3462-3473 (2013)) is a rabbit anti-human PD-L1 mAb publicly available from Sino Biological, Inc. (Beijing, P.R. China; Catalog number 10084-R015).

“Framework region” or “FR” as used herein means the immunoglobulin variable regions excluding the CDR regions.

“Isolated antibody” and “isolated antibody fragment” refers to the purification status and in such context means the named molecule is substantially free of other biological molecules such as nucleic acids, proteins, lipids, carbohydrates, or other material such as cellular 10 debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat,” as used herein, means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

An “anti-PD-1 antibody” useful in the any of the treatment methods, compositions and uses of the present invention include monoclonal antibodies (mAb), or antigen binding fragments thereof, which specifically bind to human PD-1. Alternative names or synonyms for PD-1 and its ligands include: PDCD1, PD1, CD279 and SLEB2 for PD-1; PDCDILI, PDL1, B7H1, B7-4, CD274 and B7-H for PD-L1; and PDCD1L2, PDL2, B7-DC, Btdc and CD273 for PD-L2. In any of the treatment methods, compositions and uses of the present invention in which a human individual is being treated, the PD-1 antibody or antigen binding fragment thereof is a PD-1 antagonist that blocks binding of human PD-L1 to human PD-1, or blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. An anti-PD-1 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

“PD-L1” or “PD-L2” expression means any detectable level of expression of the designated PD-L protein on the cell surface or of the designated PD-L mRNA within a cell or tissue, unless otherwise defined. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and real-time quantitative RT-PCR.

Several approaches have been described for quantifying PD-L1 protein expression in IHC assays of tumor tissue sections. See, e.g., Thompson et al., PNAS 101 (49): 17174-17179 (2004); Thompson et al., Cancer Res. 66:3381-3385 (2006); Gadiot et al., Cancer 117:2192-2201 (2011); Taube et al., Sci Transl Med 4, 127ra37 (2012); and Toplian et al., New Eng. J Med. 366 (26): 2443-2454 (2012).

One approach employs a simple binary endpoint of positive or negative for PD- L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes.

The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is >5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

A tissue section from a tumor that has been stained by IHC with a diagnostic PD- L1 antibody may also be scored for PD-L1 protein expression by assessing PD-L1 expression in both the tumor cells and infiltrating immune cells in the tissue section using a scoring process.

See WO 2014/165422. One PD-L1 scoring process comprises examining each tumor nest in the tissue section for staining, and assigning to the tissue section one or both of a modified H score (MHS) and a modified proportion score (MPS). To assign the MHS, four separate percentages are estimated across all of the viable tumor cells and stained mononuclear inflammatory cells in all of the examined tumor nests: (a) cells that have no staining (intensity=0), (b) weak staining (intensity=1+), (c) moderate staining (intensity=2+) and (d) strong staining (intensity=3+). A cell must have at least partial membrane staining to be included in the weak, moderate or strong staining percentages. The estimated percentages, the sum of which is 100%, are then input into the formula of 1 x (percent of weak staining cells)+2 x (percent of moderate staining cells)+3 x (percent of strong staining cells), and the result is assigned to the tissue section as the MHS.

The MPS is assigned by estimating, across all of the viable tumor cells and stained mononuclear inflammatory cells in all of the examined tumor nests, the percentage of cells that have at least partial membrane staining of any intensity, and the resulting percentage is assigned to the tissue section as the MPS. In some embodiments, the tumor is designated as positive for PD-L1 expression if the MHS or the MPS is positive.

Another method for scoring/quantifying PD-L1 expression in a tumor is the “combined positive score” or “CPS,” which refers to an algorithm for determining a PD-L1 expression score from a tumor sample of a patient. The CPS is useful in selecting patients for treatment with particular treatment regimens including methods of treatment comprising administration of an anti-PD-1 antibody in which expression of PD-L1 is associated with a higher response rate in a particular patient population relative to same patient population that does not express PD-L1.

The CPS is determined by determining the number of viable PD-L1 positive tumor cells, the number of viable PD-L1 negative tumor cells, and the number of viable PD-L1 positive mononuclear inflammatory cells (MIC) in a tumor tissue from a patient having a tumor and calculating the CPS using the following formula:

$$\frac{( \# P D - L 1 positive tumor cells ) + ( \# P D - L 1 positive MIC )}{( \# P D - L 1 positive tumor cells ) + ( PD - L 1 negative tumor cells ) .} \times 100 \%$$

Yet another scoring method for PD-L1 expression is the “TPS” or “tumor proportion score,” which is the percentage of tumor cells expressing PD-L1 on the cell membrane. TPS typically includes the percentage of neoplastic cells expressing PD-L1 at any intensity (weak, moderate, or strong), which can be determining using an immunohistochemical assay using a diagnostic anti-human PD-L1 mAb, e.g. antibody 20C3 and antibody 22C3, described, supra.

Cells are considered to express PD-L1 if membrane staining is present, including cells with partial membrane staining.

The level of PD-L1 mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some preferred embodiments, PD-L1 expression in a tumor sample is determined to be elevated if PD-L1 protein (and/or PD-L1 mRNA) in the sample is at least 10%, 20%, or 30% greater than in the control.

“Tissue section” refers to a single part or piece of a tissue sample, e.g., a thin slice of tissue cut from a sample of a normal tissue or of a tumor.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Variable regions” or “V region” as used herein means the segment of IgG chains which is variable in sequence between different antibodies. It extends to Kabat residue 109 in the light chain and 113 in the heavy chain.

“RECIST 1.1 Response Criteria” as used herein means the definitions set forth in Eisenhauer, E. A. et al., Eur. J. Cancer 45:228-247 (2009) for target lesions or non-target lesions, as appropriate based on the context in which response is being measured.

### II. PD-I Antibodies and Antigen Binding Fragments Useful in the Invention

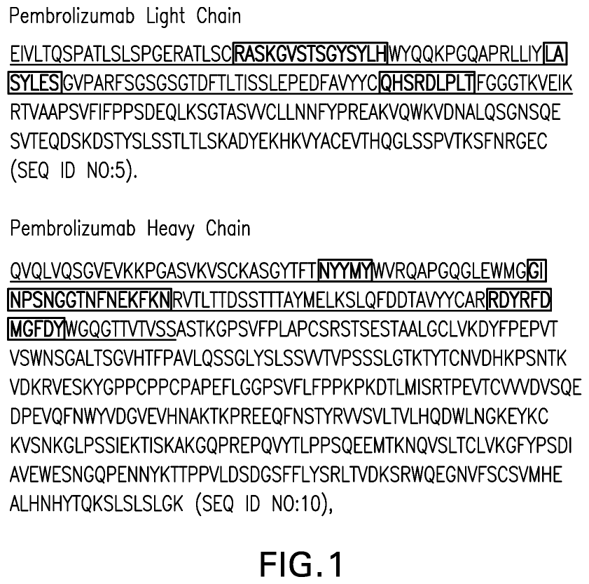

Examples of mAbs that bind to human PD-1, useful in the treatment methods and uses of the invention, are described in U.S. Pat. Nos. 7,521,051, 8,008,449, and 8,354,509. Specific anti-human PD-1 mAbs useful as a PD-1 antagonist in the treatment methods, compositions, and uses of the present invention include: pembrolizumab (formerly known as MK-3475, SCH 900475 and lambrolizumab), a humanized IgG4 mAb with the structure described in WHO Drug Information, Vol. 27, No. 2, pages 161-162 (2013) and which comprises the heavy and light chain amino acid sequences shown in FIG. 1, and the humanized antibodies h409AI 1, h409A16 and h409A17, which are described in WO 2008/156712 and in Table 3.

In some embodiments of the treatment methods, compositions, kits and uses of the present invention, the anti-PD-1 antibody, or antigen binding fragment thereof, comprises: (a) light chain CDRs comprising a sequence of amino acids as set forth in SEQ ID NOs: 1, 2 and 3 and heavy chain CDRs comprising a sequence of amino acids as set forth in SEQ ID NOs: 6, 7 and 8; or (b) light chain CDRs comprising a sequence of amino acids as set forth in SEQ ID NOs: 11, 12 and 13 and heavy chain CDRs comprising a sequence of amino acids as set forth in SEQ ID NOs: 14, 15 and 16. In some embodiments, the anti-PD-1 antibody or antigen binding fragment thereof is a human antibody. In other embodiments, the anti-PD-1 antibody or antigen binding fragment thereof is a humanized antibody. In other embodiments, the anti-PD-1 antibody or antigen binding fragment thereof is a chimeric antibody. In specific embodiments, the anti-PD-1 antibody or antigen binding fragment thereof is a monoclonal antibody.

In other embodiments of the treatment methods, compositions, kits and uses of the present invention, the anti-PD-1 antibody, or antigen binding fragment thereof, specifically binds to human PD-1 and comprises (a) a heavy chain variable region comprising an amino acid sequence as set forth in SEQ ID NO:9, or a variant thereof, and (b) a light chain variable region comprising an amino acid sequence selected from the group consisting of SEQ ID NO:4 or a variant thereof; SEQ ID NO:22 or a variant thereof; and SEQ ID NO:23 or a variant thereof.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region.

A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

In another embodiment of the treatment methods, compositions, kits and uses of the present invention, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO: 10, or a variant thereof; and (b) a light chain comprising or consisting of a sequence of amino acids as set forth in SEQ ID NO:5, or a variant thereof; SEQ ID NO:24, or a variant thereof; or SEQ ID NO:25, or a variant thereof.

In yet another embodiment of the treatment methods, compositions and uses of the invention, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:10 and (b) a light chain comprising or consisting of a sequence of amino acids as set forth in SEQ ID NO:5.

Table 3 and Table 4 below provides a list of the amino acid sequences of exemplary anti-PD-1 mAbs for use in the treatment methods, compositions, kits and uses of the present invention.

TABLE 3
Exemplary anti-human PD-1 antibodies
A. Comprises light and heavy chain CDRs of hPD-1.09A in WO2008/156712 (light and
heavy chain CDRs of pembrolizumab)

| | |
|---|---|
| CDRL1 | RASKGVSTSGYSYLH |
| | SEQ ID NO: 1 |
| CDRL2 | LASYLES |
| | SEQ ID NO: 2 |
| CDRL3 | QHSRDLPLT |
| | SEQ ID NO: 3 |
| CDRH1 | NYYMY |
| | SEQ ID NO: 6 |
| CDRH2 | GINPSNGGTNFNEKFKN |
| | SEQ ID NO: 7 |
| CDRH3 | RDYRFDMGFDY |
| | SEQ ID NO: 8 |

B. Comprises light and heavy chain CDRs of hPD-1.08A in WO2008/156712

| | |
|---|---|
| CDRL1 | RASKSVSTSGFSYLH |
| | SEQ ID NO: 11 |
| CDRL2 | LASNLES |
| | SEQ ID NO: 12 |
| CDRL3 | QHSWELPLT |
| | SEQ ID NO: 13 |
| CDRH1 | SYYLY |
| | SEQ ID NO: 14 |
| CDRH2 | GVNPSNGGTNFSEKFKS |
| | SEQ ID NO: 15 |
| CDRH3 | RDSNYDGGFDY |
| | SEQ ID NO: 16 |

C. Comprises the mature hl 09 A heavy chain variable (VH) region and one of the mature
K09A light chain variable (VL) regions in WO 2008/156712

| | |
|---|---|
| Heavy chain VH | QVQLVQSGVEVKKPGASVKVSCKASGYTFTNYYMYWVRQA |
| | PGQGLEWMGGINPSNGGTNFNEKFKNRVTLTTDSSTTTAYME |
| | LKSLQFDDTAVYYCARRDYRFDMGFDYWGQGTTVTVSS |
| | SEQ ID NO: 9 (VH of pembrolizumab) |
| Light chain VL | EIVLTQSPATLSLSPGERATLSCRASKGVSTSGYSYLHWYQQK |
| | PGQAPRLLIYLASYLESGVPARFSGSGSGTDFTLTISSLEPEDFA |
| | VYYCQHSRDLPLTFGGGTKVEIK |
| | SEQ ID NO: 4 (VL of pembrolizumab) or |
| | EIVLTQSPLSLPVTPGEPASISCRASKGVSTSGYSYLHWYLQKP |
| | GQSPQLLIYLASYLESGVPDRFSGSGSGTDFTLKISRVEAEDVG |
| | VYYCQHSRDLPLTFGQGTKLEIK |
| | (SEQ ID NO: 22) or |
| | DIVMTQTPLSLPVTPGEPASISCRASKGVSTSGYSYLHWYLQK |
| | PGQSPQLLIYLASYLESGVPDRFSGSGSGTAFTLKISRVEAEDV |
| | GLYYCQHSRDLPLTFGQGTKLEIK |
| | SEQ ID NO: 23 |
| Heavy chain | QVQLVQSGVEVKKPGASVKVSCKASGYTFTNYYMYWVRQA |
| | PGQGLEWMGGINPSNGGTNFNEKFKNRVTLTTDSSTTTAYME |
| | LKSLQFDDTAVYYCARRDYRFDMGFDYWGQGTTVTVSSAST |
| | KGPSVFPLAPCSRSTSESTAALGCLVKDYFPEPVTVSWNSGAL |
| | TSGVHTFPAVLQSSGLYSLSSVVTVPSSSLGTKTYTCNVDHKP |
| | SNTKVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKTIS |
| | KAKGQPREPQVYTLPPSQEEMTKNQVSLTCLVKGFYPSDIAVE |
| | WESNGQPENNYKTTPPVLDSDGSFFLYSRLTVDKSRWQEGNV |
| | FSCSVMHEALHNHYTQKSLSLSLGK |
| | SEQ ID NO: 10 (heavy chain of pembrolizumab) |
| Light chain | EIVLTQSPATLSLSPGERATLSCRASKGVSTSGYSYLHWYQQK |
| | PGQAPRLLIYLASYLESGVPARFSGSGSGTDFTLTISSLEPEDFA |
| | VYYCQHSRDLPLTFGGGTKVEIKRTVAAPSVFIFPPSDEQLKSG |
| | TASVVCLLNNFYPREAKVQWKVDNALQSGNSQESVTEQDSK |
| | DSTYSLSSTLTLSKADYEKHKVYACEVTHQGLSSPVTKSFNRG |
| | EC |
| | SEQ ID NO: 5 (light chain of pembrolizumab) or |
| | EIVLTQSPLSLPVTPGEPASISCRASKGVSTSGYSYLHWYLQKP |
| | GQSPQLLIYLASYLESGVPDRFSGSGSGTDFTLKISRVEAEDVG |
| | VYYCQHSRDLPLTFGQGTKLEIKRTVAAPSVFIFPPSDEQLKSG |
| | TASVVCLLNNFYPREAKVQWKVDNALQSGNSQESVTEQDSK |
| | DSTYSLSSTLTLSKADYEKHKVYACEVTHQGLSSPVTKSFNRG |
| | EC |
| | SEQ ID NO: 24 or |
| | DIVMTQTPLSLPVTPGEPASISCRASKGVSTSGYSYLHWYLQK |
| | PGQSPQLLIYLASYLESGVPDRFSGSGSGTAFTLKISRVEAEDV |
| | GLYYCQHSRDLPLTFGQGTKLEIKRTVAAPSVFIFPPSDEQLKS |
| | GTASVVCLLNNFYPREAKVQWKVDNALQSGNSQESVTEQDS |
| | KDSTYSLSSTLTLSKADYEKHKVYACEVTHQGLSSPVTKSFNR |
| | GEC |
| | SEQ ID NO: 25 |

TABLE 4
Nivolumab Light Chain

| | |
|---|---|
| CDR1 | RASQSVSSYLA |
| | (SEQ ID NO: 26) |
| CDR2 | DASNRAT |
| | (SEQ ID NO: 27) |
| CDR3 | QQSSNWPRT |
| | (SEQ ID NO: 28) |
| Variable | EIVLTQSPATLSLSPGERATLSCRASQSVSSYLAWYQQKPG |
| Region | QAPRLLIYDASNRATGIPARFSGSGSGTDFTLTISSLEPED |
| | FAVYYCQQSSNWPRTFGQGTKVEIK |
| | (SEQ ID NO: 29) |
| Light | EIVLTQSPATLSLSPGERATLSCRASQSVSSYLAWYQQKPG |
| Chain | QAPRLLIYDASNRATGIPARFSGSGSGTDFTLTISSLEPED |
| | FAVYYCQQSSNWPRTFGQGTKVEIKRTVAAPSVFIFPPSDE |
| | QLKSGTASVVCLLNNFYPREAKVQWKVDNALQSGNSQESVT |
| | EQDSKDSTYSLSSTLTLSKADYEKHKVYACEVTHQGLSSPV |
| | TKSFNRGEC (SEQ ID NO: 30) |

Nivolumab Heavy Chain

| | |
|---|---|
| CDR1 | NSGMH |
| | (SEQ ID NO: 31) |
| CDR2 | VIWYDGSKRYYADSVKG |
| | (SEQ ID NO: 32) |
| CDR3 | NDDY |
| | (SEQ ID NO: 33) |
| Variable | QVQLVESGGGVVQPGRSLRLDCKASGITFSNSGMHWVRQAP |
| Region | GKGLEWVAVIWYDGSKRYYADSVKGRFTISRDNSKNTLFLQ |
| | MNSLRAEDTAVYYCATNDDYWGQGTLVTVSS |
| | (SEQ ID NO: 34) |
| Heavy | QVQLVESGGGVVQPGRSLRLDCKASGITFSNSGMHWVRQAP |
| Chain | GKGLEWVAVIWYDGSKRYYADSVKGRFTISRDNSKNTLFLQ |
| | MNSLRAEDTAVYYCATNDDYWGQGTLVTVSSASTKGPSVFP |
| | LAPCSRSTSESTAALGCLVKDYFPEPVTVSWNSGALTSGVH |
| | TFPAVLQSSGLYSLSSVVTVPSSSLGTKTYTCNVDHKPSNT |
| | KVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKT |
| | ISKAKGQPREPQVYTLPPSQEEMTKNQVSLTCLVKGFYPSD |
| | IAVEWESNGQPENNYKTTPPVLDSDGSFFLYSRLTVDKSRW |
| | QEGNVFSCSVMHEALHNHYTQKSLSLSLGK |
| | (SEQ ID NO: 35) |

### III. Methods and Uses of the Invention

The present invention provides a method of treating metastatic triple negative breast cancer in a human patient which has been identified as having a PD-L1 enriched tumor, the 5 method comprising administering to the patient, as monotherapy, an anti-PD-i antibody, or antigen binding fragment thereof, wherein the PD-L1 enriched tumor is a tumor identified as having a CPS score of ≥10. In one aspect, the method of treating metastatic triple negative breast cancer (mTNBC) comprises administering to the patient, as monotherapy, an anti-PD1 antibody, or antigen binding fragment thereof, wherein the PD-L1 enriched tumor is a tumor identified as having a CPS score of≥10 before the anti-PD1 antibody is administered.

Compositions, uses, and kits for use with the methods described herein also form part of the invention. In some embodiments of any of the methods, compositions, kits and uses described herein, the PD-L1 enriched tumor is a tumor identified as having a CPS score of ≥20.

In some embodiments of any of the methods and uses described herein, the method comprises administering (i) about 200 mg of an anti-PD-1 antibody or antigen binding fragment thereof to the patient every approximately three weeks or (ii) about 400 mg of an anti-PD-1 antibody, or antigen binding fragment thereof, to the patient every approximately six weeks.

In some embodiments of any of the methods, compositions, kits and uses described herein, the anti-PD-1 antibody or antigen binding fragment thereof comprises: (a) light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 6, 7 and 8; or (b) light chain CDRs comprising a sequence of amino acids as set forth in SEQ ID NOs: 11, 12 and 13 and heavy chain CDRs comprising a sequence of amino acids as set forth in SEQ ID NOs: 14, 15 and 16.

In some embodiments of any of the methods, compositions, kits and uses described herein, the anti-PD-1 antibody or antigen-binding fragment thereof comprises (a) a heavy chain variable region comprising a sequence of amino acids as set forth in SEQ ID NO:9, or a variant of SEQ ID NO:9, and (b) a light chain variable region comprising: (i) a sequence of amino acids as set forth in SEQ ID NO:4, or a variant of SEQ ID NO:4, (ii) a sequence of amino acids as set forth in SEQ ID NO:22, or a variant of SEQ ID NO:22, or (iii) a sequence of amino acids as set forth in SEQ ID NO:23, or a variant of SEQ ID NO:23.

In some embodiments of any of the methods, compositions, kits and uses described herein, the anti-PD-1 antibody or antigen-binding fragment thereof comprises a heavy chain variable region comprising a sequence of amino acids as set forth in SEQ ID NO:9 and a light chain variable region comprising a sequence of amino acids as set forth in SEQ ID NO:4 In some embodiments of any of the methods, compositions, kits and uses described herein, the anti-PD-1 antibody or antigen-binding fragment thereof is a monoclonal antibody comprising (a) a heavy chain comprising a sequence of amino acids as set forth in SEQ ID NO:10, or a variant of SEQ ID NO:10, and (b) a light chain comprising a sequence of amino acids as set forth in SEQ ID NO:5, a variant of SEQ ID NO:5, SEQ ID NO:24, a variant of SEQ ID NO:24, SEQ ID NO:25, or a variant of SEQ ID NO:25.

In some embodiments of any of the methods, compositions, kits and uses described herein, the anti-PD-1 antibody, or antigen binding fragment thereof, is a monoclonal antibody comprising a heavy chain comprising a sequence of amino acids as set forth in SEQ ID NO:10 and a light chain comprising a sequence of amino acids as set forth in SEQ ID NO:5.

In particular embodiments of any of the methods, compositions, kits and uses described herein, the anti-PD-1 antibody, or antigen- binding fragment thereof, is pembrolizumab.

In some embodiments of any of the methods, compositions, kits and uses described herein, the anti-PD-1 antibody or antigen binding fragment thereof comprises: (a) light chain complementarity determining regions (CDRs) comprising a sequence of amino acids as set forth in SEQ ID NOs: 26, 27, and 28 and heavy chain CDRs comprising a sequence of amino acids as set forth in SEQ ID NOs: 31, 32 and 33.

In some embodiments of any of the methods, compositions, kits and uses described herein, the anti-PD-1 antibody or antigen-binding fragment thereof comprises (a) a heavy chain variable region comprising a sequence of amino acids as set forth in SEQ ID NO:34, or a variant of SEQ ID NO:34, and (b) a light chain variable region comprising: (i) a sequence of amino acids as set forth in SEQ ID NO:29, or a variant of SEQ ID NO:29.

In some embodiments of any of the methods, compositions, kits and uses described herein, the anti-PD-1 antibody or antigen-binding fragment thereof comprises a heavy chain variable region comprising a sequence of amino acids as set forth in SEQ ID NO:35 and a light chain variable region comprising a sequence of amino acids as set forth in SEQ ID NO:30.

In particular embodiments of any of the methods, compositions, kits and uses described herein, the anti-PD-1 antibody, or antigen- binding fragment thereof, is nivolumab. In some embodiments of any of the methods and uses described herein, the method comprises administering (i) about 240 mg of an anti-PD-1 antibody (e.g., nivolumab) or antigen binding fragment thereof to the patient every approximately two weeks or (ii) about 480 mg of an anti-PD-1 antibody(e.g., nivolumab), or antigen binding fragment thereof, to the patient every approximately four weeks.

In some embodiments of any of the methods and uses described herein, the patient has received at least one prior systemic treatment for mTNBC before receiving anti-PD-1 monotherapy as described herein. In other embodiments of any of the methods and uses described herein, the patient has received at least two prior systemic treatments for mTNBC before receiving anti-PD-1 monotherapy as described herein. In some embodiments of any of the methods and uses described herein, the patient has disease progression following the at least one prior systemic treatment. In other embodiments of any of the methods and uses described herein, the patient has disease progression following the at least two prior systemic treatments. In other embodiments of any of the methods and uses described herein, the prior treatment comprises treatment with an anthracycline and/or taxane in the neoadjuvant, adjuvant, or metastatic setting.

In embodiments of any of the methods, compositions, kits and uses described herein, the anti-PD-1 antibody is an anti-PD-1 antibody, or antigen binding fragment thereof, as supra in Section II, entitled “PD-1 Antibodies and Antigen Binding Fragments Useful in the Invention”. In particular embodiments of any of the methods, compositions, kits and uses described herein, the anti-PD-1 antibody is pembrolizumab. In other embodiments, the anti-PD-1 antibody is a variant of pembrolizumab.

In all of the methods, compositions, kits and uses described herein, the anti-PD-1 antibody or antigen-binding fragment inhibits the binding of PD-L1 to PD-1, and preferably also inhibits the binding of PD-L2 to PD-1. In some preferred embodiments of the treatment methods, compositions, kits and uses of the invention, the anti-PD-1 antibody or antigen-binding fragment is a monoclonal antibody, which specifically binds to PD-1 and blocks the binding of PD-L1 to PD- 1.

In some embodiments of any of the methods and uses described herein, the anti-PD-1 antibody, or antigen binding fragment thereof, is administered to the patient about once every three weeks for 12 weeks or more. In other embodiments of any of the methods and uses described herein, the anti-PD-1 antibody, or antigen binding fragment thereof is administered to the patient once every three weeks for 15 weeks or more, 18 weeks or more, 21 weeks or more, 24 weeks or more, 27 weeks or more, 30 weeks or more, 33 weeks or more, 36 weeks or more, 39 weeks or more, 42 weeks or more, 45 weeks or more, or 48 weeks or more.

In some embodiments of any of the methods and uses described herein, the anti-PD-1 antibody, or antigen binding fragment thereof, is administered to the patient about once every six weeks for 12 weeks or more. In other embodiments of any of the methods and uses described herein, the anti-PD-1 antibody, or antigen binding fragment thereof is administered to the patient once every six weeks for 18 weeks or more, 24 weeks or more, 30 weeks or more, 36 weeks or more, 42 weeks or more, 48 weeks or more.

In any of any of the methods, compositions, kits and uses described herein, the anti-PD-1 antibody or antigen binding fragment is any of the antibodies or antigen-binding fragments described in Section II of the Detailed Description of the Invention “PD-1 Antibodies and Antigen Binding Fragments Useful in the Invention” herein. In some embodiments of any of the methods, compositions, kits and uses described herein, the anti-PD-1 antibody is pembrolizumab or an antigen-binding fragment thereof, or an antibody which cross competes with pembrolizumab for binding to human PD-1. In some embodiments of any of the methods, compositions, kits and uses described herein, the anti-PD-1 antibody is a variant of pembrolizumab; i.e. an antibody or antigen-binding fragment having light chain CDRs comprising a sequence of amino acids as set forth in SEQ ID NOs: 1, 2 and 3 and heavy chain CDRs comprising a sequence of amino acids as set forth in SEQ ID NOs: 6, 7 and 8.

In some embodiments of any of the methods, compositions, kits and uses described herein, the anti-PD-1 antibody is nivolumab or an antigen-binding fragment thereof In some embodiments of any of the methods, compositions, kits and uses described herein, the anti-PD-1 antibody is a variant of nivolumab; i.e. an antibody or antigen-binding fragment having light chain CDRs comprising a sequence of amino acids as set forth in SEQ ID NOs: 26, 27 and 28 and heavy chain CDRs comprising a sequence of amino acids as set forth in SEQ ID NOs: 31, 32 and 33

In embodiments of any of the methods and uses described herein, a patient is administered an intravenous (IV) infusion of a medicament comprising any of the anti-PD-I antibodies or antigen-binding fragments described herein.

In alternative embodiments of any of the methods and uses described herein, the patient is administered (e.g., by a clinician) or administers any of the anti-PD-I antibodies or antigen-binding fragments subcutaneously.

### IV. Compositions and Kits

The invention also relates to compositions comprising a dosage of an anti-PD-1 antibody (e.g., pembrolizumab) or antigen binding fragment thereof and a pharmaceutically acceptable carrier or excipient, for use in treating a patient which has been identified as having a PD-L1 enriched tumor (e.g., a CPS score of≥10 or a CPS score of ≥20). The anti-PD-1 antibody may be produced, for example, in CHO cells using conventional cell culture and recovery/purification technologies.

In some embodiments, a composition comprising an anti-PD-1 antibody as a PD-1 antagonist may be provided as a liquid formulation or prepared by reconstituting a lyophilized powder with sterile water for injection prior to use. WO 2012/135408 describes the preparation of liquid and lyophilized medicaments comprising pembrolizumab that are suitable for use in the present invention.

In embodiments of the invention, the composition further comprises histidine buffer at about pH 5.0 to pH 6.0. In particular embodiments, the histidine is present in a concentration of about 10 mM.

In the compositions of the invention, the anti-PD-1 antibody or antigen binding fragment thereof can be any of the antibodies and antigen binding fragments described herein, i.e. described in Section II of the Detailed Description of the Invention “PD-1 Antibodies and Antigen Binding Fragments Useful in the Invention” (e.g. pembrolizumab). In some embodiments, the PD-1 antibody or antigen binding fragment can be as otherwise disclosed herein.

The invention also relates to a kit for treating a patient with metastatic triple negative breast cancer which patient has been identified has having a PD-L1 enriched tumor (e.g., of a CPS score of ≥10 or a CPS score of≥20), the kit comprising: (a) 200 mg or 400 mg of an anti-PD-1 antibody or antigen binding fragment thereof, and (b) instructions for using the anti-PD-1 antibody or antigen binding fragment thereof in any of the methods for treating a mTNBC as described herein.

In any of the kits of the invention, the PD-1 antibody or antigen binding fragment can be any of the antibodies or antigen-binding fragments described in Section II of the Detailed Description of the Invention “PD-1 Antibodies and Antigen Binding Fragments Useful in the Invention”. In some embodiments, the PD-1 antibody or antigen binding fragment can be as otherwise disclosed herein.

The kits of the invention may provide the anti-PD-1 antibody or antigen-binding fragments thereof in a container and a package insert. The container contains at least one dose (e.g., about 200 mg or about 400 mg) of a medicament comprising an anti-PD-1 antibody, or antigen binding fragment thereof, and the package insert, or label, which comprises instructions for treating a patient with mTNBC using the medicament in accordance with any of the methods for treating a mTNBC as described herein, including that the medicament is intended for use in treating a patient having a mTNBC, wherein the tumor expresses PD-L1 by, e.g., an IHC assay. In another embodiment, at least one of the patient's tumors expresses PD-L1 with a CPS of ≥10%. In another embodiment, at least one of the patient's tumors expresses PD- L1 with a CPS of ≥20%. The container may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes.

These and other aspects of the invention, including the exemplary specific embodiments listed below, will be apparent from the teachings contained herein.

### GENERAL METHODS

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Productsfor Life Science Research, St. Louis, Mo.; pp. 45-89; Amersham Pharmacia Biotech (2001) BioDirectory, Piscataway, N.J., pp. 384-391).

Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Shepherd and Dean (eds.) (2000)Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J Immunol. 146:169-175; Gibellini et al. (1998) J Immunol. 160:3891-3898; Hsing and Bishop (1999) J Immunol. 162:2804-2811; Everts et al. (2002) J Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma- Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, PA; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, VECTOR NTI Suite (Informax, Inc, Bethesda, Md.); GCG Wisconsin Package (Accelrys, Inc., San Diego, Calif.); DECYPHER (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J Biochem. 133:17-21; von Heijne (1986) Nucleic Acids Res. 14:4683-4690).

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1

A Phase 3 Study of Pembrolizumab versus Single-Agent Chemotherapy for Metastatic Triple-Negative Breast Cancer.

This study is an international, randomized, open-label, phase 3 study designed to compare PFS and OS between pembrolizumab and single-agent chemotherapy as second-line or third-line therapy in patients with mTNBC (NCT02555657).

### Study Design

A cohort of participants are randomly assigned 1:1 to receive either (i) pembrolizumab 200 mg Q3W or (ii) investigator choice of capecitabine, eribulin, gemcitabine, vinorelbine, with a maximum enrollment cap of 60% total enrollment for each chemotherapy drug. Randomization is stratified by PD-L1 tumor status and history of prior neoadjuvant/adjuvant therapy versus de novo metastatic disease at initial diagnosis.

Eligible patients have recurrent mTNBC; 1 or 2 prior systemic treatments for mTNBC; documented disease progression on/after most recent therapy; previous treatment with an anthracycline and/or a taxane in the neoadjuvant/adjuvant or metastatic setting and ECOG PS 0-1. Eligible patients also have provision of a tumor sample for determination of triple-negative status and PD-L1 expression.

Patients are followed up for safety (<90 days) and survival (every 3 months). Response was assessed every 9 weeks for 1 year and every 12 weeks thereafter. Primary end points were OS in the PD-L1 positive tumors (CPS ≥10), OS in patients with PD-L1 positive tumors (CPS ≥1), and OS in all patients. Key secondary endpoints include PFS in all patients, ORR in all patients, and safety and tolerability. Additional secondary end points include DCR and DOR in all patients and patients with PD-L1 positive tumors (CPS ≥1 or CPS ≥10). Exploratory end points include OS, PFS, ORR, and DOR in patients with PD-L1 positive tumors using additional CPS cutpoints. PD-L1 expression was assessed at a central laboratory using the PD-L1 IHC 22C3 pharmDx (Agilent Technologies) assay defined as the combined positive score (CPS).

Specifically, it was assessed centrally in newly obtained core or excisional biopsy from metastatic, not previously irradiated, tumor lesion. ORR was assessed per RECIST v1.1 by blinded, independent central review.

### Statistical Considerations

Planned enrollment of 600 patients v. actual enrollment of 622 patients. Overall alpha for study: strictly controlled at one-sided a=2.5%. Final analysis (reviewed by independent data monitoring committee) was 24 months after enrollment complete with 334 OS events in CPS >1. The purpose was to evaluate superiority of pembrolizumab monotherapy v. chemotherapy for OS. OS superiority of pembrolizumab monotherapy v. chemotherapy was tested in all patients only if superiority was shown in patients with CPS ≥1.

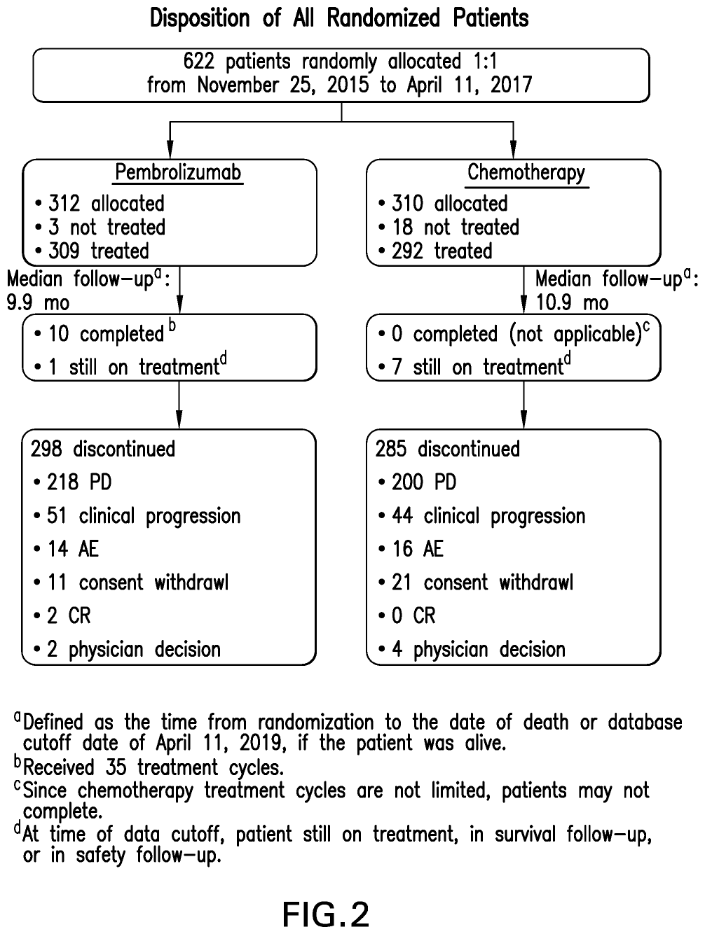

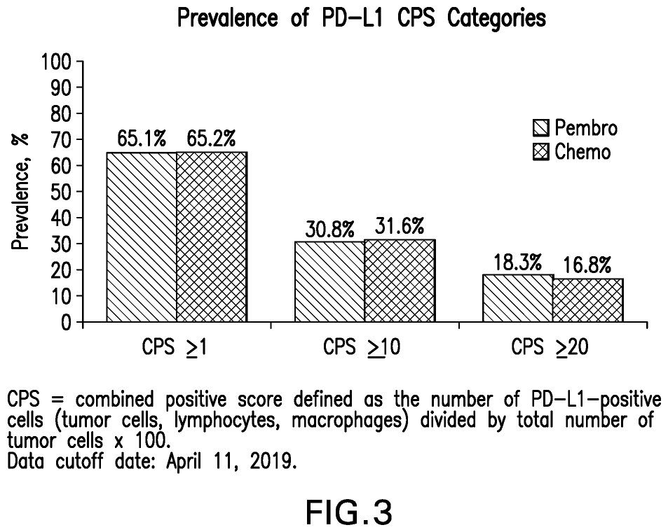

The disposition of all randomized patients is set forth in FIG. 2. The prevalence of PD-L1 CPS for the pembrolizumab and the chemotherapy patient populations is set forth in FIG. 3. The baseline characteristics of the pembrolizumab and chemotherapy patient populations is set forth in Table 5 below (data cut off Apr. 11, 2019).

TABLE 5
baseline characteristics of pembrolizumab and chemotherapy patient populations

| Characteristic, | Pembro | Chemo | Characteristic, | Pembro | Chemo |
|---|---|---|---|---|---|
| n (%) | N = 312 | N = 310 | n (%) | N = 312 | N = 310 |
| Age, median | 50 (28-85) | 50 (25-79) | Prior | 246 | 246 (79.4) |
| (range), y | | | neoadjuvant/adjuvant | (78.8) | |
| Age <65 years | 264 (84.6) | 260 (83.9) | <6 month time to | 156 | 151 (48.7) |
| Post-menopausal | 238 (76.3) | 239 (77.1) | progression on 1 L | (50.0) | |
| ECOG PS | 169 (54.2) | 158 (51.0) | ≥6 month time to | 156 | 159 (51.3) |
| Value 0 | | | progression on 1 L | (50.0) | |
| ECOG PS | 141 (45.2) | 151 (48.7) | Eribulin | — | 167 (53.9) |
| Value 1 | | | chemotherapy | | |
| 1 Prior line of | 187 (59.9) | 187 (60.3) | Capecitabine | — | 85 (27.4) |
| therapy | | | chemotherapy | | |
| 2 Prior line of | 124 (39.7) | 123 (39.7) | Vinorelbine | — | 43 (13.9) |
| therapy | | | chemotherapy | | |
| | | | Gemcitabine | — | 15 (4.8) |
| | | | chemotherapy | | |

Of 1098 participants screened, 622 were allocated to treatment with pembrolizumab (N=312) or chemotherapy (N=310) between Nov. 25, 2015 and Apr. 11, 2017 at 150 sites in 31 countries. Of those, 601 participants were treated and 21 never received study medication.

After a median time from randomization to data cutoff of 31.4 months (interquartile range, 27.8-34.4) for the pembrolizumab arm and 31.5 months (interquartile range, 27.8-34.6) for the chemotherapy arm, 298/312 (95.5%) participants discontinued pembrolizumab and 285/310 (91.9%) participants discontinued chemotherapy; the most common reason for discontinuation in both groups was disease progression. Disposition was similar between all participants and participants with PD-L1-positive CPS ≥1 tumors, PD-L1-positive CPS ≥10 tumors, and in the exploratory cohort of PD-L1-positive CPS ≥20 tumors.

Baseline demographics were well balanced in both treatment groups (see Table 6 below). Most participants were female (99.7%), median age was 52.0 years (range, 25-85), and 46.9% had an ECOG PS of 1. Nearly all participants (99.8%) had received prior therapy. Overall, 65.1% of participants had PD-L1-positive CPS ≥1 tumors, 31.2% had PD-L1-positive CPS ≥10 tumors, and 17.5% had PD-L1-positive CPS ≥20 tumors. Disease characteristics were representative of patients with advanced/metastatic triple-negative breast cancer. Baseline demographics and disease characteristics of participants with PD-L1-positive CPS ≥1 tumors, PD-L1-positive CPS ≥10 tumors, and PD-L1-positive CPS ≥20 tumors were generally consistent with those of all participants (see Table 6 below).

TABLE 6
Demographic and disease characteristics at baseline*

| | | |
|---|---|---|
| | Pembrolizumab | Chemotherapy |
| Characteristic | N = 312 | N = 310 |

Age

| | | |
|---|---|---|
| Median (range)-yr | 50 (28-85) | 53 (25-79) |
| <65 yr-no. (%) | 264 (84.6) | 260 (83.9) |
| Female sex-no. (%) | 312 (100.0) | 308 (99.4) |

Geographic region-no. (%)

| | | |
|---|---|---|
| Europe | 143 (45.8) | 180 (46.2) |
| Asia | 83 (26.6) | 98 (31.6) |
| North America | 39 (12.5) | 21 (6.8) |
| Australia | 7 (2.2) | 16 (5.2) |
| Rest of world | 40 (12.8) | 36 (11.6) |

Menopausal status-no. (%)

| | | |
|---|---|---|
| Pre-menopausal | 74 (23.7) | 69 (22.3) |
| Post-menopausal | 238 (76.3) | 239 (77.1) |
| Not applicable | 0 | 2 (0.6) |

PD-L1 status^{†}-no. (%)

| | | |
|---|---|---|
| CPS ≥1 | 203 (65.1) | 202 (65.2) |
| CPS <1 | 109 (34.9) | 108 (34.8) |
| CPS ≥10 | 96 (30.8) | 98 (31.6) |
| CPS <10 | 216 (69.2) | 212 (68.4) |
| CPS ≥20 | 57 (18.3) | 52 (16.8) |
| CPS <20 | 255 (81.7) | 258 (83.2) |

EGOG performance status

| | | |
|---|---|---|
| 0 | 169 (54.2) | 158 (51.0) |
| 1 | 141 (45.2) | 151 (48.7) |
| ≥2 | 1 (0.3) | 0 |
| Missing | 1 (0 3) | 1 (0.3) |

Lactase dehydrogenase-no. (%)

| | | |
|---|---|---|
| Normal | 149 (47.8) | 153 (49.4) |
| Elevated > ULN and <2 · 5 ULN | 154 (49.4) | 148 (47.7) |
| Elevated ≥2 · 5 ULN | 8 (2.6) | 7 (2.3) |
| Missing | 1 (0.3) | 2 (0.6) |

History of brain metastases-no. (%)

| | | |
|---|---|---|
| Yes | 20 (6.4) | 22 (7.1) |
| No | 292 (93.6) | 288 (92.9) |

Prior neoadjuvant/adjuvant therapy vs de novo metastatic disease-no. (%)

| | | |
|---|---|---|
| Prior neoadjuvant/adjuvant therapy | 246 (78.8) | 246 (79.4) |
| De novo metastatic disease | 66 (21.2) | 64 (20.6) |

Number of prior lines of therapy for recurrent/metastatic disease-no. (%)

| | | |
|---|---|---|
| 0 | 1 (0.3) | 0 |
| 1 | 187 (59.9) | 187 (60.3) |
| 2 | 124 (39.7) | 123 (39.7) |

Time to progression on first line therapy-no. (%)

| | | |
|---|---|---|
| <6 months | 156 (50.0) | 151 (48.7) |
| ≥6 months | 156 (50.0) | 159 (51.3) |

*Based on data from the intention-to-treat population. Percentages may not total 100 because of rounding. CPS = combined positive score. PD-L1 = programmed death ligand 1. ECOG = Eastern Cooperative Oncology Group.
^{†}The PD-L1 combined positive score was defined as number of PD-L1-positive cells (tumor cells, lymphocytes, and macrophages) divided by total number of tumor cells × 100. PD-L1 positivity was defined as CPS ≥1.

### Results

As of ii APR 2019, median follow-up was 9.9 months for pembrolizumab monotherapy (n=312) and 10.9 months for chemotherapy (n=310). Pembrolizumab did not significantly improve OS in patients with CPS ≥10 (P=0.0574) or CPS ≥1 (P=0.0728) or in all patients, although the pembrolizumab treatment effect increased as CPS increased (HR 0.78, 0.86, and 0. 97, respectively; See Table 7 below). In an exploratory analysis of patients with a PD-L1 CPS score of ≥20, median OS was 14.9 month with pembrolizumab monotherapy as compared to 12.5 months with chemotherapy (HR 0.58, 95% o CI 0.38-0.88). Pembrolizumab did not improve PFS (See Table 7 below). Duration of Response (DOR) was longer with pembrolizumab monotherapy vs chemotherapy (see Table 7). Grade 3-5 treatment-related AE rates were 1400 with pembrolizumab monotherapy (1 death) vs 36% o with chemotherapy (2 deaths). Rates of grade 3-4 immune-mediated AEs and infusion reactions were 3.2% o vs 1.00%, with no deaths (see Table 8).

TABLE 7

| | CPS ≥20 | | CPS ≥10 | |
|---|---|---|---|---|
| | Pembrolizumab | Chemotherapy | Pembrolizumab | Chemotherapy |
| | N = 57 | N = 52 | N = 96 | N = 98 |
| OS, mo, median | 14.9 | 12.5 | 12.7 | 11.6 |
| (95% CI) | (10.7-19.8) | (7.3-15.4) | (9.9-16.3) | (8.3-13.7) |
| OS, HR | 0.58 | | 0.78 | |
| (95% CI) | (0.38-0.88) | | (0.57-1.06) | |
| PFS, mo, | 3.4 | 2.4 | 2.1 | 3.4 |
| median | (2.1-4.2) | (2.1-4.1) | (2.0-2.5) | (2.3-4.1) |
| (95% CI) | | | | |
| PFS, HR | 0.76 | | 1.14 | |
| (95% CI) | (0.49-1.18) | | (0.82-1.59) | |
| ORR, % | 26.3% | 11.5% | 17.7 | 9.2 |
| (95% CO) | | | (10.7-26.8) | (4.3-16.7) |
| DOR, mo, | NR | 7.1 | NR | 7.1 |
| median (range) | (2.8-32.5+) | (4.2-25.9+) | (2.2-32.5+) | (3.8-25.9+) |
| DCR, % | | | 19.8 | 17.3 |
| (95% CI) | | | (12.4-29.2) | (10.4-26.3) |
| | CPS ≥1 | | ITT | |
| | Pembrolizumab | Chemotherapy | Pembrolizumab | Chemotherapy |
| | N = 203 | N = 202 | N = 312 | N = 310 |
| OS, mo, median | 10.7 | 10.2 | 9.9 | 10.8 |
| (95% CI) | (9.3-12.5) | (7.9-12.6) | (8.3-11.4) | (9.1-12.6) |
| OS, HR | 0.86 | | 0.97 | |
| (95% CI) | (0.69-1.06) | | (0.82-1.15) | |
| PFS, mo, | 2.1 | 3.1 | 2.1 | 3.3 |
| median | (2.0-2.1) | (2.3-4.0) | (2.0-2.1) | (2.7-4.0) |
| (95% CI) | | | | |
| PFS, HR | 1.35 | | 1.60 | |
| (95% CI) | (1.08-1.68) | | (1.33-1.92) | |
| ORR, % | 12.3 | 9.4 | 9.6 | 10.6 |
| (95% CO) | (8.1-17.6) | (5.8-14.3) | (6.6-13.4) | (7.4-14.6) |
| DOR, mo, | 12.2 | 6.5 | 12.2 | 8.3 |
| median (range) | (2.2-32.5+) | (2.1+-33.0+) | (2.2-32.5+) | (2.1+-33.0+) |
| DCR, % | 14.3 | 15.8 | 12.2 | 18.7 |
| (95% CI) | (9.8-19.9) | (11.1-21.6) | (8.8-16.3) | (14.5-23.5) |

TABLE 8
Summary of Adverse Events

| | Pembrolizumab | Chemotherapy |
|---|---|---|
| | N = 309 | N = 292 |
| All Cause | 283 (91.6%) | 279 (95.5%) |
| Grade 3 | 107 (34.6%) | 143 (49.0%) |
| Led to death | 9 (2.9%) | 9 (3.1%) |
| Led to discontinuation | 14 (4.5%) | 16 (5.5%) |
| Led to dose | 64 (20.7%) | 132 (45.2%) |
| modification^{a} | | |
| Immune-mediated and | 48 (15.5%) | 9 (3.1%) |
| infusion reactions | | |
| Grade 3-5 | 10 (3.2%) | 3 (1.0%) |
| Led to death | 0 | 0 |

^{a}Defined as drug interrupted or drug withdrawn for pembrolizumab and as dose reduced, drug interrupted or drug withdrawn for chemotherapy.

Pembrolizumab monotherapy did not significantly improve OS as 2/3L treatment for mTNBC as compared to chemotherapy in the primary analysis populations. Pembrolizumab showed a clear trend in improved efficacy with PD-L1 enrichment. Responses to pembrolizumab were more durable than those to chemotherapy. Pembrolizumab was well tolerated and had less high-grade toxicity as compared to chemotherapy.

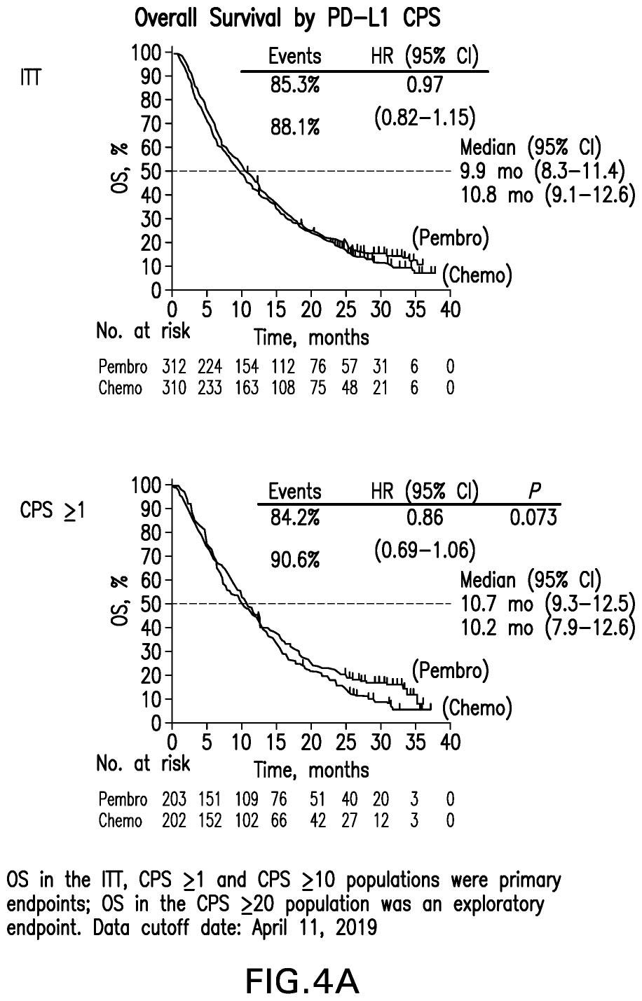

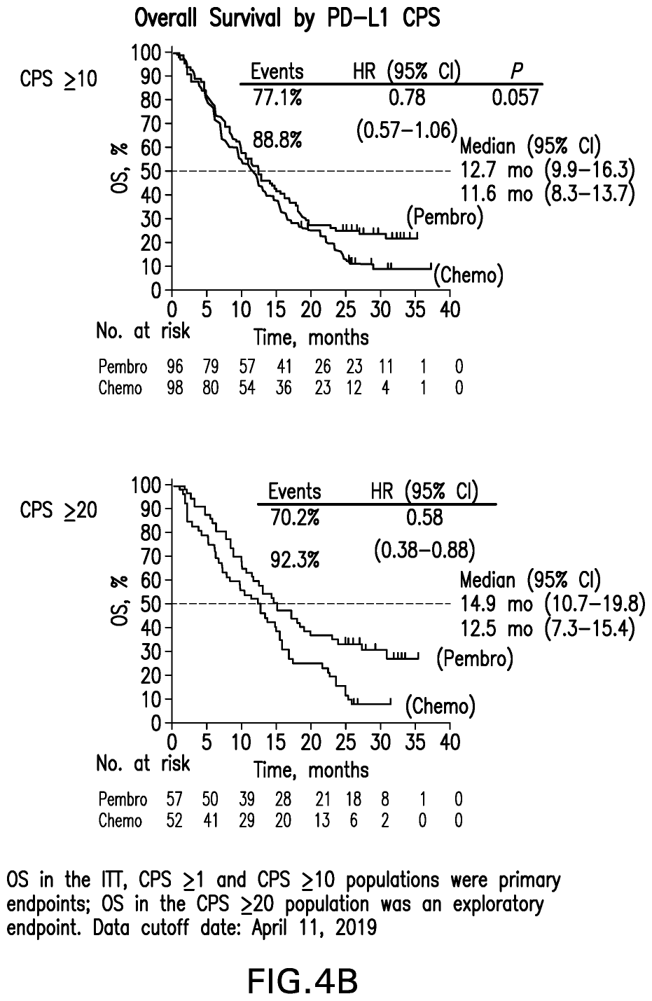

In the population of patients with PD-L1-positive CPS 10 tumors, median overall 10 survival was 12.7 months (95% CI: 9.9 to 16.3) for the pembrolizumab group and 11.6 months (95% CI: 8.3 to 13.7) for the chemotherapy group (see FIG. 4B), and the overall survival rates were 52.1% and 48.9%, respectively, at 12 months and 25.0% and 1⁶0.2% o, respectively, at 24 months. In the population of patients with PD-L1-positive CPS ≥1 tumors, median overall survival was 10.7 months (95% CI: 9.3 to 12.5) for the pembrolizumab group and 10.2 months (95% CI: 7.9 to 12.6) for the chemotherapy group (see FIG. 4A), and the overall survival rates were 45.6% and 44.7%, respectively, at 12 months and 21.3% and 15.6% o, respectively, at 24 months. The hazard ratios (pembrolizumab versus chemotherapy) were 0.78 (95% CI: 0.57 to 1.06; p=0.0574) for the population with PD-L1-positive CPS ≥10 tumors and 0.86 (95% CI: 0.86 to 1.06; p=0.0728) for the population with PD-L1-positive CPS ≥1 tumors. In the total population, median overall survival was 9.9 months for the pembrolizumab group and 10.8 months for the chemotherapy group (see FIG. 4B), and the overall survival rates were 42.8% and 45.6%, respectively, at 12 months and 20.9% and 18.9%, respectively, at 24 months. The hazard ratio for overall survival (pembrolizumab versus chemotherapy) was 0.97 (95% CI: 0.82 to 1.15) for all participants. In an exploratory analysis of overall survival in participants with PD-L1-positive CPS ≥20 tumors, median overall survival was 14.9 months (95% CI: 10.7 to 19.8) for the pembrolizumab group and 12.5 months (95% CI: 7.3 to 15.4) for the chemotherapy group (FIG. 4B), with a hazard ratio of 0.58 (95% CI: 0.38 to 0.88).

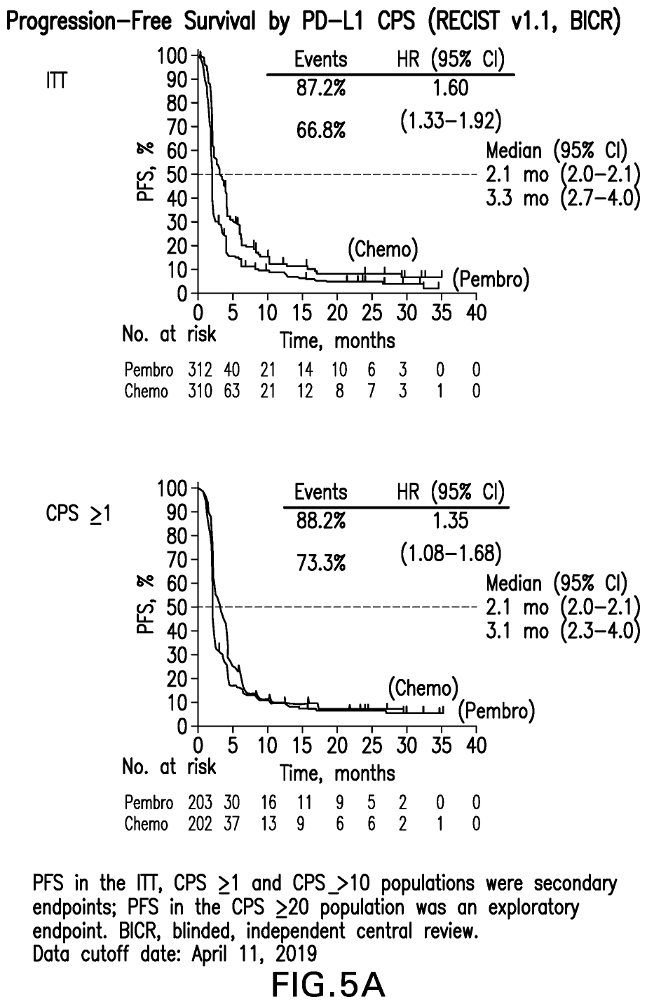

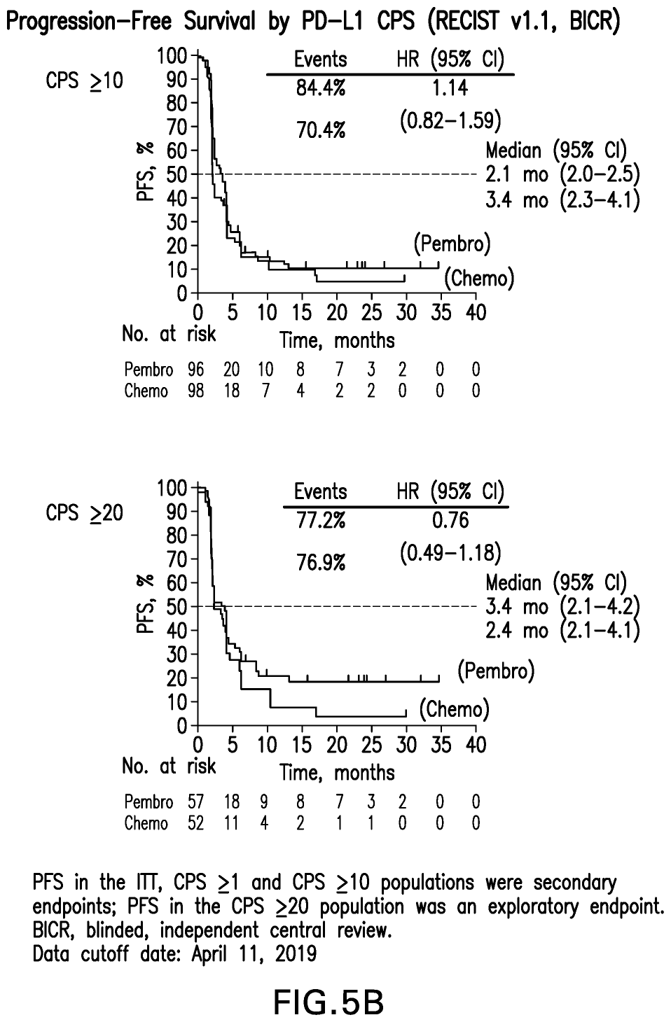

Median progression-free survival was 2.1 months (95% CI: 2.0 to 2.1) for pembrolizumab and 3.3 months (95% CI: 2.7 to 4.0) for chemotherapy, and the rates at 12 months were 8.7% versus 12.3%, respectively, with a hazard ratio (pembrolizumab versus chemotherapy) of 1.60 (95% CI: 1.33 to 1.92; see FIG. 5A). Consistent with the results for overall survival, pembrolizumab showed improved progression-free survival with PD-L1 enrichment (see FIGS. 5A-5B), with hazard ratios (pembrolizumab versus chemotherapy) of 0.76, 1.14, and 1.35 in participants with PD-L1-positive CPS ≥20 tumors, PD-L1-positive CPS ≥10 tumors, and PD-L1-positive CPS ≥1 tumors, respectively.

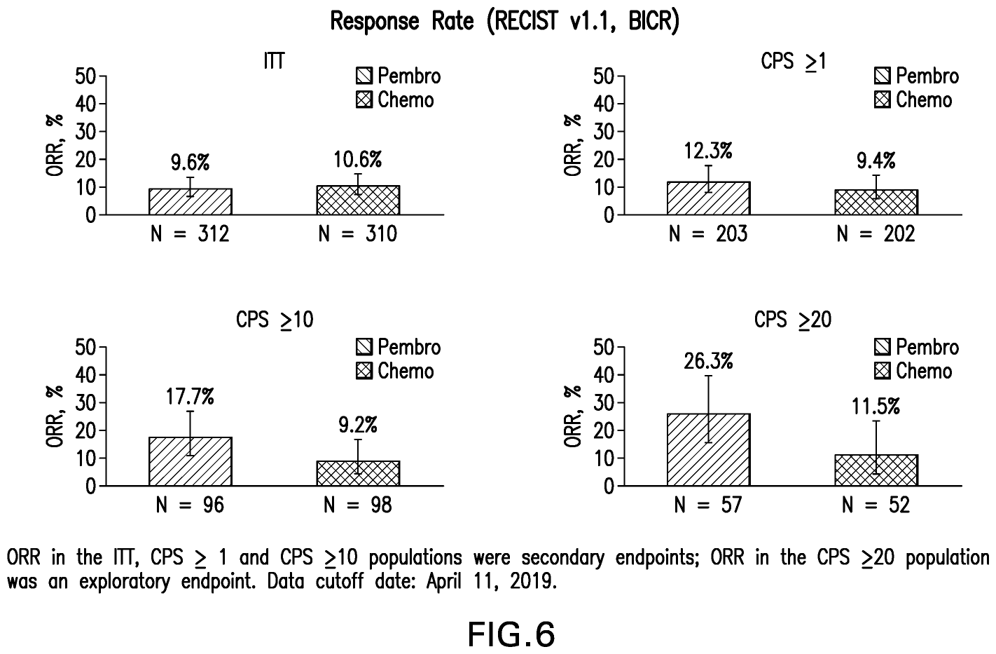

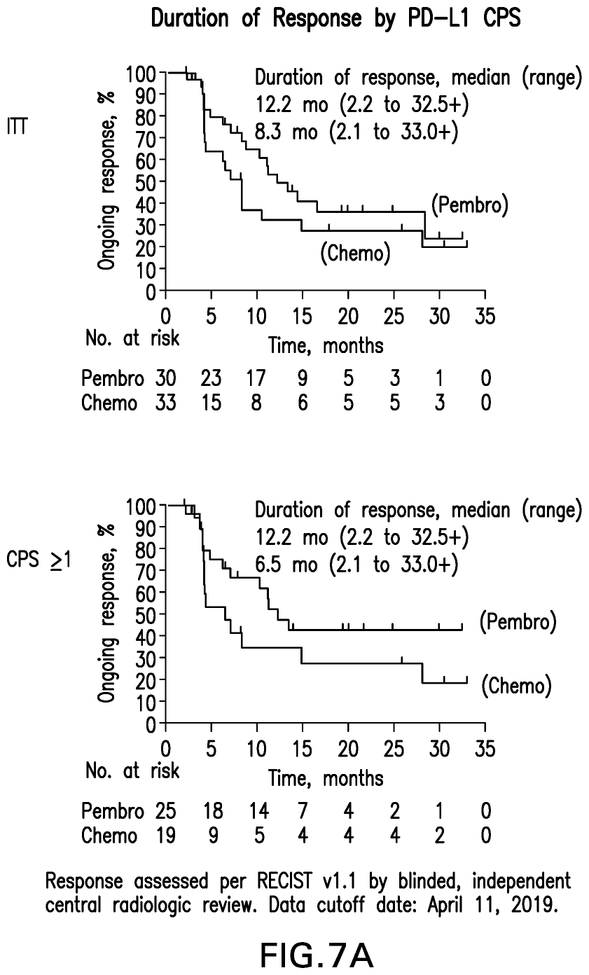

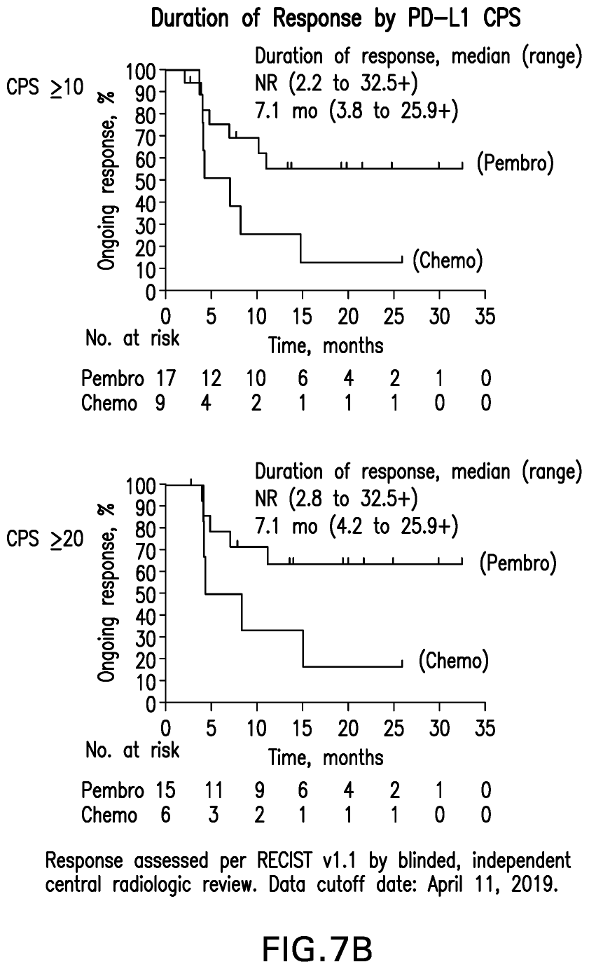

The objective response rate was 9.6% (95% CI: 6.6 to 13.4) in the pembrolizumab group and 10.6% (95% CI: 7.4 to 14.6) in the chemotherapy group (see FIG. 6 and Table 9 below). Confirmed complete responses were reported in 11 (3.5%) participants in the pembrolizumab group and 19 (6.1%) participants in the chemotherapy group. Pembrolizumab showed an improved objective response rate with PD-L1 enrichment, with rates for pembrolizumab and chemotherapy of 12.3% versus 9.4% in the population with PD-L1-positive CPS ≥1 tumors, 17.7% versus 9.2% in the population with PD-L1-positive CPS ≥10 tumors, and 26.3% versus 11.5% in the population with PD-L1-positive CPS ≥20 tumors (see FIGS. 7A-7B). In the total population, the median duration of response was 12.2 months (95% CI: 8.3 to 28.4 months) in the pembrolizumab group and 833 months (95% CI: 4.2 to 14.9 months) in the chemotherapy group; 23 of 30 responders (79.4%) in the pembrolizumab group and 15 of 33 responders (63.7%) in the chemotherapy group had an estimated duration of response ≥6 months (see Table 9 below). There was a longer duration of response with increasing PD-L1 enrichment (see FIGS. 7A-7B).

TABLE 9
Antitumor activity assessed by RECIST v1.1 per independent central review

| | Pembrolizumab | Chemotherapy |
|---|---|---|
| Antitumor Activity | N = 312 | N = 310 |
| ORR, n (%) [95% CI] | 30 (9.6) [6.6-13.4] | 33 (10.6) [7.4-14.6] |
| DCR*, n (%) [95% CI] | 38 (12.2) [8.8-16.3] | 58 (18.7) [14.5-23.5] |
| Best overall response, n (%) | | |
| Complete response | 11 (3.5) | 5 (1.6) |
| Partial response | 19 (6.1) | 28 (9.0) |
| Stable disease | 62 (19.9) | 119 (38.4) |
| No evidence of disease^{†} | 0 | 2 (0.6) |
| Progressive disease | 185 (59.3) | 95 (30.6) |
| Not able to be evaluated^{‡} | 8 (2.6) | 7 (2.3) |
| Not able to be assessed^{¶} | 27 (8.7) | 54 (17.4) |
| Time to response months, | 2.2 (1.3-13.4) | 2.1 (1.3-10.1) |
| median (range) | | |
| Duration of response, ^{∥,}** | 12.2 (8.3-28.4) | 8.3 (4.2-14.9) |
| months, median (95% CI) | | |
| Estimated rate of response | 79.4 | 63.7 |
| duration ≥6 months, ^{∥,}** % | | |
| Estimated rate of response | 53.3 | 32.4 |
| duration ≥12 months, ^{∥,}** % | | |

NR = not reached.
RECIST = Response Evaluation Criteria in Solid Tumors.
“+” indicates there is no progressive disease by the time of last disease assessment.
*DCR = the proportion of participants with complete or partial response or stable disease for ≥24 weeks.
^{†}Includes participants with no evidence of disease at baseline and at least one post-baseline time point.
^{‡}Participants who had ≥1 postbaseline tumor assessment, none of which were evaluable.
^{¶}Participants who had no postbaseline tumor assessment because of death, withdrawal of consent, loss to follow-up, or start of new anticancer therapy.

Of the 622 participants enrolled, 601 received at least one dose of study treatment and were evaluated for safety. Median time on treatment was 62 days in the pembrolizumab group and 73 days in the chemotherapy group. There was a lower incidence of treatment-related AEs in the pembrolizumab group compared with the chemotherapy group (62.1% versus 82.9%, respectively), including those that were grade 3-5 (13.9% versus 36.0%, respectively). Three participants had treatment-related AEs that led to death (circulatory collapse [n=l] in the pembrolizumab group, and pancytopenia and sepsis [n=l] and hemothorax [n=l] in the chemotherapy group). Ten (3.9% o) participants in the pembrolizumab group and 9 (3.1% o) participants in the chemotherapy group discontinued treatment because of treatment-related AEs. The treatment-related AEs that occurred in ≤10% of participants were fatigue (12.0 %) and nausea (10.0%) in the pembrolizumab group, and nausea (21.6%), neutropenia (20.5%), diarrhea (15.4), decreased neutrophil count (14.7%), fatigue (13.7%), alopecia (13.4%), anemia (13.4%), palmar-plantar erythrodysesthesia syndrome (12.3%), and decreased appetite (11.6%) in the chemotherapy group (see Table 10 below).

TABLE 10
Summary of adverse events

| | Pembrolizumab | | Chemotherapy | |
|---|---|---|---|---|
| | N = 309 | | N = 292 | |
| | Any Grade | Grade ≥3 | Any Grade | Grade ≥3 |
| Event | Number of patients (percent) | | | |
| Any adverse event* | 283 (91.6) | 107 (34.6) | 279 (95.5) | 143 (49.0) |
| Treatment-related | 192 (62.1) | 43 (13.9) | 242 (82.9) | 105 (36.0) |
| adverse event^{†} | | | | |
| Fatigue | 37 (12.0) | 3 (1.0) | 40 (13.7) | 3 (1.0) |
| Nausea | 31 (10.0) | 0 | 63 (21.6) | 0 |
| Diarrhea | 17 (5.5) | 2 (0.6) | 45 (15.4) | 8 (2.7) |
| Decreased appetite | 14 (4.5) | 0 | 34 (11.6) | 0 |
| Anemia | 13 (4.2) | 3 (1.0) | 39 (13.4) | 10 (3.4) |
| Alopecia | 2 (0.6) | 0 | 39 (13.4) | 0 |
| Neutrophil count | 2 (0.6) | 1 (0.3) | 43 (14.7) | 29 (9.9) |
| decreased | | | | |
| Palmar-plantar | 2 (0.6) | 1 (0.3) | 36 (12.3) | 7 (2.4) |
| erythrodysesthesia | | | | |
| Neutropenia | 1 (0.3) | 0 | 60 (20.5) | 39 (13.4) |
| Adverse event of | 47 (15.2) | 10 (3.2) | 8 (2.7) | 3 (1.0) |
| interest^{‡} | | | | |
| Hypothyroidism | 24 (7.8) | 1 (0.3) | 4 (1.4) | 0 |
| Hyperthyroidism | 11 (3.6) | 0 | 0 | 0 |
| Pneumonitis | 6 (1.9) | 3 (1.0) | 0 | 0 |
| Severe skin reaction | 5 (1.6) | 2 (0.6) | 1 (0.3) | 1 (0.3) |
| Adrenal insufficiency | 3 (1.0) | 1 (0.3) | 0 | 0 |
| Myositis | 2 (0.6) | 2 (0.6) | 0 | 0 |
| Colitis | 1 (0.3) | 0 | 2 (0.7) | 1 (0.3) |
| Myasthenic syndrome | 1 (0.3) | 0 | 0 | 0 |
| Nephritis | 1 (0.3) | 0 | 0 | 0 |
| Thyroiditis | 1 (0.3) | 0 | 1 (0.3) | 0 |
| Type 1 diabetes | 1 (0.3) | 1 (0.3) | 1 (0.3) | 1 (0.3) |
| mellitus | | | | |

*Listed are all adverse events that occurred during randomly allocated study treatment or within the 30 days thereafter (within 90 days for serious events). The as-treated population included all participants who underwent randomization and received ≥1 dose of study treatment. Events are listed in descending order of frequency in the pembrolizumab group. The severity of adverse events was graded according to the National Cancer Institute Common Terminology Criteria for Adverse Events, version 4.0.
^{†}Adverse events that were attributed to study treatment by the investigator. Treatment-related adverse events that occurred in at least 10% of participants or those that were considered medically relevant are reported. Participants may have had more than one event. Grade 5 treatment-related events adverse were circulatory collapse (N = 1) in the pembrolizumab group and sepsis and pancytopenia (N = 1) and hemothorax (N = 1) in the chemotherapy group.
^{‡}Adverse events based on a list of terms specified by the sponsor and considered regardless of treatment attribution by the investigator that occurred in any participant are reported.

Immune-mediated AEs, considered regardless of attribution to treatment by the investigator, occurred in 47 (15.2%) participants in the pembrolizumab group and 8 (2.7%) participants in the chemotherapy group, with rates of grade 3-4 events of 3.2% vs 1.0%, respectively (see Table 10). The most common immune-mediated AEs in the pembrolizumab and chemotherapy groups were hypothyroidism (7.8% versus 1.4%, respectively) and hyperthyroidism (3.6% versus 0%, respectively; table 7). Infusion reactions occurred in 0.3% of the pembrolizumab group and 0.3% of the chemotherapy group; none of these reactions were of grade 3 or higher.

In the present study, the safety profile of pembrolizumab in metastatic triple-negative breast cancer was generally consistent with the established safety profile of pembrolizumab monotherapy. There were fewer treatment-related AEs of any grade and of grade ≥3 in the pembrolizumab group compared with the chemotherapy group. In addition, there were no new immune-mediated AEs causally related to pembrolizumab identified.

Pembrolizumab monotherapy as second- or third-line treatment for metastatic triple-negative breast cancer demonstrated directionally favorable improvement in overall survival compared with chemotherapy in PD-L1-positive tumors (CPS 10 and CPS ≥1), but superiority to chemotherapy across all tumors was not statistically demonstrated. Since overall survival did not differ significantly between the treatment groups in patients with PD-L1-positive tumors (CPS ≥1), the statistical testing hierarchy did not allow for formal testing of overall survival in all participants. However, there was a trend toward improved overall survival with increasing tumor PD-L1 expression levels with pembrolizumab. Exploratory analyses of PD-L1-positive CPS 20 tumors provided additional support for this trend. As with overall survival, pembrolizumab did not improve progression-free survival, objective response rate, or disease control rate compared with chemotherapy in all participants, but the improved pembrolizumab treatment effect with increasing tumor PD-L1 expression was maintained across these efficacy endpoints. Responses to pembrolizumab, although relatively infrequent in most subgroups examined, were durable. Notably, the response duration in the pembrolizumab group also increased with higher PD-L1 expression, with the median duration of response not reached in participants with PD-L1-positive CPS ≥1 and PD-L1-positive CPS ≥10 tumors. By comparison, the efficacy of chemotherapy was independent of tumor PD-L1 expression, suggesting that PD-L1 expression, as defined by the tumor CPS, is a predictive biomarker for pembrolizumab-derived clinical benefit in patients with metastatic triple-negative breast cancer.

The present findings of enhanced efficacy and durability of pembrolizumab with PD-L1 enrichment are consistent with results from other studies of pembrolizumab monotherapy for the treatment of metastatic triple-negative breast cancer. In the phase 1b KEYNOTE-012 trial of pembrolizumab as first-line or greater treatment for participants with PD-L1-positive metastatic triple-negative breast cancer (N=32), there was a trend toward enhanced clinical benefit with increasing tumor PD-L1 expression, although the small sample size and enrolment of only participants with PD-L1-positive tumors precluded a definitive conclusion on the predictive role of PD-L1 (Nanda et al. Pembrolizumab in patients with advanced triple-negative breast cancer: phase Ib KEYNOTE-012 study. J Clin Oncol 2016; 34: 2460-7). Pembrolizumab monotherapy was also tested in the phase II KEYNOTE-086 trial in participants with metastatic triple-negative breast cancer (Adams et al. Pembrolizumab monotherapy for previously untreated, PD-L1-positive, metastatic triple-negative breast cancer: cohort B of the phase II KEYNOTE-086 study.

Ann Oncol 2019; 30: 405-11; Adams et al. Pembrolizumab monotherapy for previously treated metastatic triple-negative breast cancer: cohort A of the phase II KEYNOTE-086 study. Ann Oncol 2019; 30: 397-404). In participants with previously treated disease, the objective response rate was modest and independent of PD-L1 status; however, a trend toward a greater clinical benefit with pembrolizumab for disease-control rate and a longer duration of response was observed in participants with PD-L1-positive tumors versus those with PD-L1-negative tumors (Adams et al. Pembrolizumab monotherapy for previously treated metastatic triple-negative breast cancer: cohort A of the phase II KEYNOTE-086 study. Ann Oncol 2019; 30: 397-404).

The association of PD-L1 expression with treatment response in metastatic triple-negative breast cancer has also been evaluated for other single-agent immune checkpoint inhibitors. In an expansion cohort of a phase Ia trial (N=116), atezolizumab monotherapy was associated with higher response rates in participants with PD-L1-positive metastatic triple-negative breast cancer compared to participants with PD-L1-negative disease (Schmid et al. Atezolizumab in metastatic TNBC (mTNBC): Long-term clinical outcomes and biomarker analyses. American Association for Cancer Research Annual Meeting, Washington, D.C., USA; Apr. 1-5, 2017 (abstr 2986)). Additionally, a phase Ib study of avelumab monotherapy in participants with metastatic breast cancer (N=168) showed an objective response rate of 5.2% in participants with metastatic triple-negative breast cancer (N=58), which was higher in participants with PD-L1-positive versus PD-L1-negative tumors (22.2% vs. 2.6%) (Dirix et al. Avelumab, an anti-PD-L1 antibody, in patients with locally advanced or metastatic breast cancer: a phase Ib JAVELIN Solid Tumor study. Breast Cancer Res Treat 2018; 167: 671-86).

As has been seen in the treatment of triple-negative breast cancer and other tumor types, combination regimens with immunotherapy and chemotherapy have shown promising clinical benefit. In the randomized phase III IMpassioni30 trial (Schmid et al. Atezolizumab and nab-paclitaxel in advanced triple-negative breast cancer. N Engl J Med 2018; 379: 2108-21; Schmid et al. Atezolizumab plus nab-paclitaxel as first-line treatment for unresectable, locally advanced or metastatic triple-negative breast cancer (IMpassion130): updated efficacy results from a randomized, double-blind, placebo-controlled, phase 3 trial. Lancet Oncol 2020; 21: 44-59) the benefit of atezolizumab plus nab-paclitaxel versus nab-paclitaxel alone in participants with untreated metastatic triple-negative breast cancer was greatest in participants with PD-L1-positive tumors, as defined by immune cell staining ≥1% according to VENTANA PD-L1 SP142 immunohistochemical testing (Vennapusa et al. Development of a PD-L1 Complementary Diagnostic Immunohistochemistry Assay (SP142) for Atezolizumab. Appl Immunohistochem Mol Morphol 2019; 27: 92-100); in this predefined subgroup (N=369), atezolizumab plus nab-paclitaxel versus nab-paclitaxel alone resulted in a median progression-free survival of 7.5 months versus 5.0 months, a median overall survival of 25.0 months versus 18.0 months, and an objective response rate of 58.9% versus 42.6% (Schmid et al. N Engl J Med 2018; 379: 2108-21; Schmid et al. Lancet Oncol 2020; 21: 44-59). Results from the phase III KEYNOTE-355 clinical trial showed a statistically significant and clinically meaningful improvement in progression-free survival with first-line pembrolizumab combined with chemotherapy compared with chemotherapy alone in patients with metastatic triple-negative breast cancer with PD-L1-positive CPS ≥10 tumors (Cortes et al. KEYNOTE-355: Randomized, double-blind, phase III study of pembrolizumab+chemotherapy versus placebo+chemotherapy for previously untreated locally recurrent inoperable or metastatic triple-negative breast cancer. American Society of Clinical Oncology-56th Annual Meeting. (Virtual Meeting); 2020). Although the boundary for declaring a statistically significant benefit of pembrolizumab-chemotherapy in progression-free survival in patients with PD-L1-positive CPS ≥1 tumors was not crossed and formal testing in the intention-to-treat population was not performed, pembrolizumab-chemotherapy showed numerical increases in median progression-free survival in both populations and improved treatment effects over the chemotherapy control arm with PD-L1 enrichment. These findings suggest that patients with PD-L1-positive triple-negative breast cancer have a greater likelihood of clinical response to immune checkpoint inhibitors.

In the present study, the safety profile of pembrolizumab in metastatic triple-negative breast cancer was generally consistent with the established safety profile of pembrolizumab monotherapy. There were fewer treatment-related AEs of any grade and of grade ≥3 in the pembrolizumab group compared with the chemotherapy group. In addition, there were no new immune-mediated AEs causally related to pembrolizumab identified.

In conclusion, pembrolizumab monotherapy for previously treated metastatic triple-negative breast cancer did not significantly improve overall survival compared to chemotherapy in participants with PD-L1-positive tumors (CPS≥10 or CPS ≥1) or in all participants.

However, a trend toward an enriched treatment effect with increasing PD-L1 expression levels was observed with pembrolizumab. The greatest benefit with pembrolizumab was observed in an exploratory analysis of participants with strongly positive PD-L1-expressing tumors (CPS ≥20), comprising approximately 18% of the overall study population. Responses were durable with pembrolizumab and were sustained longer within a CPS-enriched population. Taken together, these findings suggest that patients with tumors with higher PD-L1 expression may have a greater likelihood of clinical benefit from pembrolizumab monotherapy.

All references cited herein are incorporated by reference to the same extent as if each individual publication, database entry (e.g. Genbank or GeneID entries), patent application, or patent, was specifically indicated to be incorporated by reference. This statement is intended by Applicants, pursuant to 37 C.F.R. § 1.57(b)(1), to relate to each and every individual publication, database entry (e.g. Genbank or GeneID entries), patent application, or patent, each of which is clearly identified in compliance with 37 C.F.R. § 1.57(b)(2), even if such citation is not immediately adjacent to a dedicated statement of incorporation by reference. Citation of the references herein is not intended as an admission that the reference is pertinent prior art, nor does it constitute any admission as to the contents or date of these publications or documents.

1. A method of treating metastatic triple negative breast cancer (mTNBC) in a human patient identified as having a PD-L1 enriched tumor, the method comprising administering to the patient, as monotherapy, an anti-PD1 antibody, or antigen binding fragment thereof, wherein the PD-L1 enriched tumor is a tumor identified as having a combined positive score (CPS) of ≥10 before the anti-PD1 antibody is administered.

2. The method of claim 1, wherein the anti-PD-1 antibody or antigen-binding fragment thereof comprises:
(a) light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 6, 7 and 8; or
(b) light chain CDRs comprising a sequence of amino acids as set forth in SEQ ID NOs: 11, 12 and 13 and heavy chain CDRs comprising a sequence of amino acids as set forth in SEQ ID NOs: 14, 15 and 16.

3. The method of claim 2, wherein the anti-PD-1 antibody or antigen-binding fragment thereof comprises:
(a) a heavy chain variable region comprising a sequence of amino acids as set forth in SEQ ID NO:9, or a variant of SEQ ID NO:9, and
(b) a light chain variable region comprising:
(i) a sequence of amino acids as set forth in SEQ ID NO:4, or a variant of SEQ ID NO:4,
(ii) a sequence of amino acids as set forth in SEQ ID NO:22, or a variant of SEQ ID NO:22, or
(iii) a sequence of amino acids as set forth in SEQ ID NO:23, or a variant of SEQ ID NO:23.

4. The method of claim 3, wherein the anti-PD-1 antibody or antigen-binding fragment thereof comprises a heavy chain variable region comprising a sequence of amino acids as set forth in SEQ ID NO:9 and a light chain variable region comprising a sequence of amino acids as set forth in SEQ ID NO:4.

5. The method of claim 4, wherein the anti-PD-1 antibody or antigen-binding fragment thereof is a monoclonal antibody comprising:
(a) a heavy chain comprising a sequence of amino acids as set forth in SEQ ID NO:10, or a variant of SEQ ID NO:10, and
(b) a light chain comprising a sequence of amino acids as set forth in SEQ ID NO:5, a variant of SEQ ID NO:5, SEQ ID NO:24, a variant of SEQ ID NO:24, SEQ ID NO:25, or a variant of SEQ ID NO:25.

6. The method of claim 1, wherein the anti-PD-1 antibody or antigen-binding fragment thereof is a monoclonal antibody comprising a heavy chain comprising a sequence of amino acids as set forth in SEQ ID NO:10 and a light chain comprising a sequence of amino acids as set forth in SEQ ID NO: 5.

7. The method of claim 1, wherein the patient has received at least one prior systemic treatment for mTNBC.

8. The method of claim 7, wherein the patient has received at least two prior systemic treatments for mTNBC.

9. The method of claim 7, wherein the patient has disease progression following the at least one prior systemic treatment.

10. The method of claim 7, wherein the at least one prior systemic treatment comprises treatment with an anthracycline and/or a taxane in the neoadjuvant, adjuvant, or metastatic setting.

11. The method of claim 1, wherein the PD-L1 enriched tumor is a tumor identified as having a CPS of ≥20.

12. The method of claim 1, wherein the anti-PD-1 antibody or antigen-binding fragment thereof is administered to the patient by intravenous or subcutaneous administration.

13. The method of claim 1, wherein the anti-PD-1 antibody or antigen-binding fragment thereof is pembrolizumab.

14. The method of claim 1, wherein the method comprises administering:
(i) about 200 mg of an anti-PD-1 antibody, or antigen binding fragment thereof, to the patient every approximately three weeks; or
(ii) about 400 mg of an anti-PD-1 antibody, or antigen binding fragment thereof, to the patient every approximately six weeks.

15. The method of claim 8, wherein the patient has disease progression following the at least two prior systemic treatments.

16. The method of claim 8, wherein the at least two prior systemic treatments comprise treatment with an anthracycline and/or a taxane in the neoadjuvant, adjuvant, or metastatic setting.
